(19) United States
(12) Patent Application Publication
(10) Pub. No.: US 2020/0165568 A1
(43) Pub. Date: May 28, 2020

(54) CELL REPROGRAMMING METHODS FOR PRODUCING CHONDROCYTES

(71) Applicant: Mogrify Limited, Cambridge (GB)

(72) Inventors: Wael Kafienah, Cambridge (GB); Julian Gough, Cambridge (GB)

(73) Assignee: Mogrify Limited, Cambridge (GB)

(21) Appl. No.: 16/625,064

(22) PCT Filed: Jun. 21, 2018
(86) PCT No.: PCT/AU2018/050617

§ 371 Date: Dec. 20, 2019

(30) Foreign Application Priority Data
Jun. 21, 2017 (AU) 2017902385

(51) Int. Cl. C12N 5/077

(52) U.S. Cl. CPC C12N 5/0655; C12N 2500/12; C12N 2501/392; A61K 45/06; C12N 2506/1307; C12N 2510/00; C12N 2501/60

(57) ABSTRACT

The present invention relates to methods and compositions for reprogramming a source cell to produce a chondrocyte, the method comprising activating or increasing the protein expression of one or more transcription factors, or variants thereof, in the source cell.

### RELATED APPLICATION

This application claims priority from Australian provisional application AU 2017902385, the entire contents of which are hereby incorporated by reference.

### FIELD OF THE INVENTION

The invention relates to methods and compositions for converting one cell type to another cell type. Specifically, the invention relates to transdifferentiation or reprogramming of a cell to a chondrocyte.

### BACKGROUND OF THE INVENTION

Articular cartilage lesions and progressive cartilage loss caused by degenerative disease are major contributors to disability in developed countries. Osteoarthritis (OA) represents a final and common pathway for all major traumatic insults to synovial joints. OA is the most common form of degenerative joint disease and a major cause of physical pain and disability. Cartilage related problems in developed society are expected to increase dramatically in the future due to the aging population and increasing incidence of obesity.

Cartilage is an avascular tissue with low metabolic activity and cell density, consisting mainly of collagen- and proteoglycan-rich extracellular matrix. Poor innate access to reparative cell sources results in low regeneration capacity of damaged cartilage.

There are currently no effective pharmacotherapies capable of restoring the original structure and function of damaged articular cartilage. This has resulted in the development of cell-based and biological therapies for treating OA and related orthopaedic disorders. Current clinical therapies are inadequate to regenerate the native hyaline cartilage structure in articulating joints, but instead produce mechanically inferior fibrocartilage highly increasing the risk of treatment failure in the long term. Moreover, novel cell sources and culture methods are needed before cell based therapies can reach their full potential.

Cell-based regenerative therapy requires the generation of specific cell types for replacing tissues damaged by injury, disease or age. Cell-replacement therapies have the potential to rapidly generate a variety of therapeutically important cell types directly from one's own easily accessible tissues, such as skin or blood. Such immunologically-matched cells would also pose less risk for rejection after transplantation. Moreover, these cells would manifest less tumorigenicity since they are terminally differentiated. In the context of OA, there are a number of clinical trials using autologous chondrocytes and autologous chondrogenic adult stem cells. These treatments are characterised by patient to patient variability, clonal variation, inconsistent potency, limited donor tissue and expensive expansion in GMP laboratories. To date, there is not a cell source capable of regenerating cartilage to its native, normal form in terms of structure, content, or organisation.

Transdifferentiation, the process of converting from one cell type to another without going through a pluripotent state, may have great promise for regenerative medicine but has yet to be reliably applied. Although it may be possible to switch the phenotype of one somatic cell type to another, the elements required for conversion are difficult to identify and in most instances unknown. The identification of factors to directly reprogram the identity of cell types is currently limited by, amongst other things, the cost of exhaustive experimental testing of plausible sets of factors, an approach that is inefficient and unscalable.

There is a need for a new and/or improved method for identifying the factors required to convert one cell type to another, and in particular, for converting a cell type to a chondrocyte which may have utility in treating conditions, for example, where chondrocyte implantation is required.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method for reprogramming a source cell, the method comprising increasing the protein expression of one or more transcription factors, or variants thereof, in the source cell, wherein the source cell is reprogrammed to exhibit at least one characteristic of a chondrocyte, wherein:
the source cell is selected from the group consisting of dermal fibroblasts embryonic stem cells, or de-differentiated chondrocytes, and
the transcription factors are one or more of those listed in Tables 1 to 4.

The present invention provides a method of generating a cell exhibiting at least one characteristic of a chondrocyte from a source cell, the method comprising:
increasing the amount of one or more transcription factors, or variants thereof, in the source cell; and
culturing the source cell for a sufficient time and under conditions to allow differentiation to a chondrocyte; thereby generating the cell exhibiting at least one characteristic of a chondrocyte from a source cell, wherein:
the source cell is selected from the group consisting of dermal fibroblasts, embryonic stem cells, and de-differentiated chondrocytes; and
the transcription factors are one or more of those listed in Table 1 to 4.

The present invention also provides a method for reprogramming a dermal fibroblast, embryonic stem cell, or de-differentiated chondrocyte, the method comprising increasing the protein expression of one or more of the transcription factors in Tables 1 to 4 or variants thereof, in the source cell, wherein the source is reprogrammed to exhibit at least one characteristic of chondrocyte.

The present invention provides a method for reprogramming a source cell to a cell that exhibits at least one characteristic of a chondrocyte comprising: i) providing a source cell, or a cell population comprising a source cell; ii) contacting said source cell with one or more agents that activate or increase the expression of one or more transcription factors; and iii) culturing said cell or cell population, and optionally monitoring the cell or cell population for at least one characteristic of a chondrocyte cell, wherein:
the source cell is selected from the group consisting of dermal fibroblasts, embryonic stem cells and de-differentiated chondrocytes;
the transcription factors are one or more of those listed in Tables 1 to 4.

The present invention provides a method for reprogramming a source cell to a cell that exhibits at least one characteristic of a chondrocyte comprising: i) providing a source cell, or a cell population comprising a source cell; ii) contacting said source cell with one or more small molecules that activate or increase the expression of one or more transcription factors; and iii) culturing said cell or cell population, and optionally monitoring the cell or cell population for at least one characteristic of a chondrocyte cell, wherein:
the source cell is selected from the group consisting of dermal fibroblasts, embryonic stem cells and de-differentiated chondrocytes;
the transcription factors are one or more of those listed in Tables 1 to 4.

Preferably, the transcription factors are one or more of SOX9, PPAR γ, BMP-2, PITX1, TGFβ3, VDR and RUNX1. More preferably, all transcription factors in the group of SOX9, PPAR γ, BMP-2, PITX1, TGFβ3, VDR and RUNX1 are activated or have increase expression resulting from the contact of the source cell with one or more small molecules.

Preferably, the small molecules for activating or increasing transcription of the transcription factors are one or more of [4-[(5,6,7,8-Tetrahydro-5,5,8,8-tetramethyl-2-naphthalenyl)carboxamido]benzoic acid (AM580), beta-estradiol, calcitriol, ciglitazone, kartogenin, Lithium chloride, melatonin, and rhosin hydrochloride. More preferably, all 8 small molecules are used.

The present invention provides a method for reprogramming a source cell to a cell that exhibits at least one characteristic of a chondrocyte comprising: i) providing a source cell, or a cell population comprising a source cell; ii) transfecting said source cell with one or more nucleic acids comprising a nucleotide sequence that encodes one or more transcription factors; and iii) culturing said cell or cell population, and optionally monitoring the cell or cell population for at least one characteristic of a chondrocyte cell, wherein:
the source cell is selected from the group consisting of dermal fibroblasts, embryonic stem cells and de-differentiated chondrocytes;
the transcription factors are one or more of those listed in Tables 1 to 4.

In any method of the invention described herein, the source cell is a fibroblast, and the target cell is a chondrocyte cell and the transcription factors are any one or more of the transcription factors listed in Tables 1 or 4.

In a preferred embodiment, all of the transcription factors listed in a single row of Table 1 may be used. Alternatively, the factors may be the combination of the transcription factors and proteins listed as follows:

### a) PPRX2, PITX1, SMAD6, FOXC1, SIX2, AHR, FOSB and JUNB;

### b) BARX1, PPRX2, SMAD6, FOXC1, SIX2, AHR, FOSB and JUNB;

### c) BARX1, PITX1, PPRX2, FOXC1, SIX2, AHR, FOSB and JUNB;

### d) BARX1, PITX1, SMAD6, PPRX2, SIX2, AHR, FOSB and JUNB;

### e) BARX1, PITX1, SMAD6, FOXC1, PPRX2, AHR, FOSB and JUNB;

### f) BARX1, PITX1, SMAD6, FOXC1, SIX2, PPRX2, FOSB and JUNB;

### g) BARX1, PITX1, SMAD6, FOXC1, SIX2, AHR, PPRX2 and JUNB; or

### h) BARX1, PITX1, SMAD6, FOXC1, SIX2, AHR, FOSB and PPRX2.

Preferably, the fibroblast is a dermal fibroblast.

In any method of the invention described herein, the source cell is an embryonic stem cell, and the target cell is a chondrocyte cell and the transcription factors are any one or more of the transcription factors listed in Table 2.

In a preferred embodiment, all of the transcription factors listed in a single row of Table 2 may be used. Alternatively, the factors may be the combination of the transcription factors and proteins listed as follows:

### a) HOXA11, PITX1, SMAD6, NFKB1, FOXC1, AHR, SIX2 and JUNB;

### b) BARX1, PRRX2, SMAD6, NFKB1, FOXC1, AHR, SIX2 and JUNB;

### c) BARX1, PITX1, TGFB3, NFKB1, FOXC1, AHR, SIX2 and JUNB;

### d) BARX1, PITX1, SMAD6, IRF1, FOXC1, AHR, SIX2 and JUNB;

### e) BARX1, PITX1, SMAD6, NFKB1, PRRX2, AHR, SIX2 and JUNB;

### f) BARX1, PITX1, SMAD6, NFKB1, FOXC1, PRRX2, SIX2 and JUNB;

### g) BARX1, PITX1, SMAD6, NFKB1, FOXC1, AHR, PRRX2 and JUNB; or

### h) BARX1, PITX1, SMAD6, NFKB1, FOXC1, AHR, SIX2 and FOSB.

Preferably, the embryonic stem cell is a human embryonic stem cell.

In any method of the invention described herein, the source cell is a de-differentiated chondrocyte, and the target cell is a chondrocyte cell and the transcription factors are any one or more of the transcription factors listed in Table 3.

In a preferred embodiment, all of the transcription factors listed in a single row of Table 3 may be used. Alternatively, the factors may be the combination of the transcription factors and proteins listed as follows:

### a) IL11, SOX5, VEGFA, RUNX1, VDR, NR2F1, FOSB and PRRX1;

### b) SOX9, TCF4, VEGFA, RUNX1, VDR, NR2F1, FOSB and PRRX1;

### c) SOX9, SOX5, HOXA7, RUNX1, VDR, NR2F1, FOSB and PRRX1;

### d) SOX9, SOX5, VEGFA, TCF4, VDR, NR2F1; FOSB and PRRX1;

### e) SOX9, SOX5, VEGFA, RUNX1, TCF4, NR2F1, FOSB and PRRX1;

### f) SOX9, SOX5, VEGFA, RUNX1, VDR, HOXA7, FOSB and PRRX1;

### g) SOX9, SOX5, VEGFA, RUNX1, VDR, NR2F2, FOSB and PRRX1;

### h) SOX9, SOX5, VEGFA, RUNX1, VDR, NR2F1, HOXA7 and PRRX1; or

### i) SOX9, SOX5, VEGFA, RUNX1, VDR, NR2F1, FOSB and HOXA7.

Preferably, the at least one characteristic of the chondrocyte is up-regulation of any one or more target cell markers and/or change in cell morphology. Relevant markers are described herein and known to those in the art. Exemplary markers for chondrocytes include: CD44, CD49, CD10, CD9, CD95, Integrin α10β1, CD105, SOX9, SMAD3, SOX5, SOX6, SMAD 5/6, the production of sulphated glycosaminoglycans (GAG), aggrecan, type II collagen (expression of COL2A1).

Typically, conditions suitable for chondrocyte differentiation include culturing the cells for a sufficient time and in a suitable medium. A sufficient time of culturing may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days. A suitable medium may be one shown in Table 6.

In any method described herein, the method may further include the step of expanding the cells exhibiting at least one characteristic of a chondrocyte to increase the proportion of cells in a population exhibiting at least one characteristic of a chondrocyte. The step of expanding the cells may be in culture for a sufficient time and under conditions for generating a population of cells as described below.

In any method described herein, the method may further include the step of administering the cells, or cell population including a cell, exhibiting at least one characteristic of a chondrocyte, to an individual.

The present invention also provides a method for preventing the de-differentiation of a primary chondrocyte. It will be understood that the approach for preventing de-differentiation mirrors the approach for reprogramming a de-differentiated cell to a re-differentiated chondrocyte. As such, any other methods described herein for re-programming a de-differentiated chondrocyte to a re-differentiated chondrocyte can also be used for preventing de-differentiation of primary chondrocytes.

The present invention also provides a cell exhibiting at least one characteristic of a chondrocyte produced by a method as described herein. The cell may be an isolated cell.

The present invention also provides a population of cells, wherein at least 5% of cells exhibit at least one characteristic of a chondrocyte and those cells are produced by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population exhibit at least one characteristic of a chondrocyte. The population of cells may be an isolated population, or substantially pure population of cells.

The present invention also relates to kits for producing a cell exhibiting at least one characteristic of a chondrocyte as disclosed herein. In some embodiments, a kit comprises one or more nucleic acids having one or more nucleic acid sequences encoding a transcription factor described herein or variant thereof.

In alternative embodiments, a kit comprises one or more small molecules for activating or increasing the expression of one or more transcription factors described herein, or variants thereof. More preferably, the kit comprises one or more of the following small molecules: [4-[(5,6,7,8-Tetrahydro-5,5,8,8-tetramethyl-2-naphthalenyl)carboxamido]benzoic acid (AM580), beta-estradiol, calcitriol, ciglitazone, kartogenin, lithium chloride, melatonin, and rhosin hydrochloride.

Preferably, the kit can be used to produce a cell exhibiting at least one characteristic of a chondrocyte. Preferably, the kit can be used with a source cell which is a dermal fibroblast, an embryonic stem cell or a de-differentiated chondrocyte. In some embodiments, the kit further comprises instructions for reprogramming a source cell to a cell exhibiting at least one characteristic of a chondrocyte according to the methods as disclosed herein. Preferably, the present invention provides a kit when used in a method of the invention described herein.

The present invention relates to a composition comprising at least one chondrocyte and at least one agent which increases the activity or the protein expression of one or more transcription factors in the chondrocyte. Further, the transcription factor may be any of the transcription factors described herein. Preferably, the transcription factors are as described in Tables 1 to 4.

In any embodiment of the present invention, the agent may be one or more of [4-[(5,6,7,8-Tetrahydro-5,5,8,8-tetramethyl-2-naphthalenyl) carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin and forskolin.

In any embodiment, a histone deacetylase inhibitor (HDAC inhibitor), or any other molecule for opening the chromatin in the source cell may be used to increase the efficacy of the small molecule agent. In one example, the molecule is the HDAC inhibitor vorinostat.

Typically, the protein expression, or amount, of a transcription factor as described herein is increased by contacting the cell with an agent which activates or increases the expression of the transcription factor. In any embodiment, the agent is selected from the group consisting of: a nucleotide sequence, a protein, an aptamer and small molecule, ribosome, RNAi agent and peptide-nucleic acid (PNA) and analogues or variants thereof. In certain embodiments, the agent is exogenous. In preferred embodiments, the agent is a small molecule. In certain embodiments, the nucleotide sequence is included as part of a transcriptional activation system (e.g., a gRNA for use in CRISPR/Cas9 systems or a TALEN) for increasing the expression of one or more transcription factors.

In some embodiments, the small molecule agent is selected from: [4-[(5,6,7,8-Tetrahydro-5,5,8,8-tetramethyl-2-naphthalenyl)carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin and forskolin.

Preferably, the small molecule agent includes a retinoic acid or an analog or derivative thereof, including any one or more of: [4-[(5,6,7,8-Tetrahydro-5,5,8,8-tetramethyl-2-naphthalenyl)carboxamido]benzoic acid (AM580), all-trans retinoic acid and 9-cis retinoic acid.

In some embodiments, a combination of small molecules is used for increasing the protein expression, or amount of, one or more transcription factors as described herein. For example, in certain embodiments, the combination of small molecules includes at least a retinoic acid or an analog or derivative thereof and calcitriol. Alternatively, the combination of small molecules includes at least [4-[(5,6,7,8-Tetrahydro-5,5,8,8-tetramethyl-2-naphthalenyl) carboxamido] benzoic acid (AM580), calcitriol, leucovorin, vorinostat and forskolin. Alternatively, the combination of small molecules may include at least (all-trans)-retinoic acid, 9-cis retinoic acid, calcitriol, leucovorin.

In certain preferred embodiments, the combination of small molecules is: [4-[(5,6,7,8-Tetrahydro-5,5,8,8-tetramethyl-2-naphthalenyl)carboxamido]benzoic acid (AM580), beta-estradiol, calcitriol, ciglitazone, kartogenin, lithium chloride, melatonin, and rhosin hydrochloride. Alternatively, the combination of small molecules is: [4-[(5,6,7,8-Tetrahydro-5,5,8,8-tetramethyl-2-naphthalenyl)carboxamido]benzoic acid (AM580), calcitriol, leucovorin, forskolin and vorinostat. Alternatively, the combination of small molecules is: all-trans retinoic acid, 9-cis retinoic acid, calcitriol, leucovorin and vorinostat.

In some embodiments, the protein expression, or amount, of a transcription factor as described herein is increased by introducing at least one nucleic acid comprising a nucleotide sequence encoding a transcription factor, or encoding a functional fragment thereof, in the cell. Preferably, the nucleotide sequence encoding a transcription factor is at least 70%, 80%, 85%, 90%, 91%, 92%, 93%, 94%, 95%, 96%, 97%, 98%, or 99% identical to a sequence with an accession number listed in Table 3.

Preferably, the nucleic acid further includes a heterologous promoter. Preferably, the nucleic acid is in a vector, such as a viral vector or a non-viral vector. Preferably, the vector is a viral vector comprising a genome that does not integrate into the host cell genome. The viral vector may be a retroviral vector or a lentiviral vector.

Any method as described herein may have one or more, or all, steps performed in vitro, ex vivo or in vivo.

In further embodiments, the present invention provides one or more of [4-[(5,6,7,8-Tetrahydro-5,5,8,8-tetramethyl-2-naphthalenyl) carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin, vorinostat and forskolin, for use in a method of treating osteoarthritis or other condition characterised by degeneration of cartilage tissue. Preferably, the invention comprises at least a retinoic acid or derivative thereof (such as [4-[(5,6,7,8-Tetrahydro-5,5,8,8-tetramethyl-2-naphthalenyl) carboxamido]benzoic acid (AM580), all-trans retinoic acid, 9-cis retinoic acid), for use in a method of treating osteoarthritis or other condition characterised by degeneration of cartilage tissue. Alternatively, the invention comprises at least calcitriol for use in a method of treating osteoarthritis or other condition characterised by degeneration of cartilage tissue. More preferably, the invention comprises at least a retinoic acid or derivative thereof and calcitriol for use in a method of treating osteoarthritis or other condition characterised by degeneration of cartilage tissue.

In further preferred embodiments the present invention provides [4-[(5,6,7,8-Tetrahydro-5,5,8,8-tetramethyl-2-naphthalenyl)carboxamido]benzoic acid (AM580), beta-estradiol, calcitriol, ciglitazone, kartogenin, lithium chloride, melatonin, and rhosin hydrochloride for use in a method of treating osteoarthritis or other condition characterised by degeneration of cartilage tissue. In addition, the present invention provides [4-[(5,6,7,8-Tetrahydro-5,5,8,8-tetramethyl-2-naphthalenyl) carboxamido]benzoic acid (AM580), calcitriol, leucovorin, forskolin and vorinostat for use in a method of treating osteoarthritis or other condition characterised by degeneration of cartilage tissue. Further still, the invention provides all-trans retinoic acid, 9-cis retinoic acid, calcitriol, leucovorin and vorinostat for use in a method of treating osteoarthritis or other condition characterised by degeneration of cartilage tissue.

The present invention also provides a method of treating osteoarthritis, or other condition characterised by degeneration of cartilage tissue, the method comprising administering to an individual in need thereof, a composition comprising one or more of [4-[(5,6,7,8-Tetrahydro-5,5,8,8-tetramethyl-2-naphthalenyl) carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin, vorinostat and forskolin, thereby treating the osteoarthritis or other condition in the individual. Preferably, the composition comprises at least a retinoic acid or derivative thereof (such as [4-[(5,6,7,8-Tetrahydro-5,5,8,8-tetramethyl-2-naphthalenyl) carboxamido]benzoic acid (AM580), all-trans retinoic acid, 9-cis retinoic acid). Alternatively, the composition comprises at least calcitriol. More preferably, the composition comprises at least a retinoic acid or derivative thereof and calcitriol.

Further, the present invention the use of one or more of [4-[(5,6,7,8-Tetrahydro-5,5,8,8-tetramethyl-2-naphthalenyl) carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin, vorinostat and forskolin, in the manufacture of a medicament for the treatment of osteoarthritis, or other condition characterised by degeneration of cartilage tissue. Preferably, the invention comprises the use of at least a retinoic acid or derivative thereof (such as [4-[(5,6,7,8-Tetrahydro-5,5,8,8-tetramethyl-2-naphthalenyl) carboxamido]benzoic acid (AM580), all-trans retinoic acid, 9-cis retinoic acid), in the manufacture of a medicament for the treatment of osteoarthritis, or other condition characterised by degeneration of cartilage tissue. Alternatively, the invention comprises the use of at least calcitriol in the manufacture of a medicament for the treatment of osteoarthritis, or other condition characterised by degeneration of cartilage tissue. More preferably, the invention comprises the use of at least a retinoic acid or derivative thereof and calcitriol.

In further preferred embodiments the present invention provides the use of [4-[(5,6,7,8-Tetrahydro-5,5,8,8-tetramethyl-2-naphthalenyl)carboxamido]benzoic acid (AM580), beta-estradiol, calcitriol, ciglitazone, kartogenin, lithium chloride, melatonin, and rhosin hydrochloride in the manufacture of a medicament for the treatment of osteoarthritis, or other condition characterised by degeneration of cartilage tissue. In addition, the present invention provides the use of [4-[(5,6,7,8-Tetrahydro-5,5,8,8-tetramethyl-2-naphthalenyl) carboxamido] benzoic acid (AM580), calcitriol, leucovorin, forskolin and vorinostat in the manufacture of a medicament for the treatment of osteoarthritis, or other condition characterised by degeneration of cartilage tissue. Further still, the invention provides the use of all-trans retinoic acid, 9-cis retinoic acid, calcitriol, leucovorin and vorinostat in the manufacture of a medicament for the treatment of osteoarthritis, or other condition characterised by degeneration of cartilage tissue.

Still further, the present invention provides a pharmaceutical composition comprising one or more of [4-[(5,6,7,8-Tetrahydro-5,5,8,8-tetramethyl-2-naphthalenyl) carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin, vorinostat and forskolin, for use in a method of treating osteoarthritis or other condition characterised by degeneration of cartilage tissue. Preferably, the pharmaceutical composition comprises at least a retinoic acid or derivative thereof (such as [4-[(5,6,7,8-Tetrahydro-5,5,8,8-tetramethyl-2-naphthalenyl) carboxamido]benzoic acid (AM580), all-trans retinoic acid, 9-cis retinoic acid). Alternatively, the pharmaceutical composition comprises at least calcitriol. More preferably, the pharmaceutical composition comprises at least a retinoic acid or derivative thereof and calcitriol.

The present invention provides a pharmaceutical composition comprising [4-[(5,6,7,8-Tetrahydro-5,5,8,8-tetramethyl-2-naphthalenyl)carboxamido]benzoic acid (AM580), beta-estradiol, calcitriol, ciglitazone, kartogenin, lithium chloride, melatonin, and rhosin hydrochloride for use in a method of treating osteoarthritis or other condition characterised by degeneration of cartilage tissue. In addition, the present invention provides a pharmaceutical composition comprising [4-[(5,6,7,8-Tetrahydro-5,5,8,8-tetramethyl-2-naphthalenyl) carboxamido] benzoic acid (AM580), calcitriol, leucovorin, forskolin and vorinostat for use in a method of treating osteoarthritis or other condition characterised by degeneration of cartilage tissue. Further still, the invention provides a pharmaceutical composition comprising all-trans retinoic acid, 9-cis retinoic acid, calcitriol, leucovorin and vorinostat for use in a method of treating osteoarthritis or other condition characterised by degeneration of cartilage tissue.

Further aspects of the present invention and further embodiments of the aspects described in the preceding paragraphs will become apparent from the following description, given by way of example and with reference to the accompanying drawings.

It will be understood that the invention disclosed and defined in this specification extends to all alternative combinations of two or more of the individual features mentioned or evident from the text or drawings. All of these different combinations constitute various alternative aspects of the invention.

### BRIEF DESCRIPTION OF THE DRAWINGS

FIG. 1: Macroscopic appearance of fibroblasts reprogrammed to chondrocytes using small molecules.

FIG. 2: Time course of fibroblast chondrocytic conversion based on SOX9 expression.

FIG. 3: Expression of SMAD signalling proteins in fibroblasts reprogrammed to chondrocytes.

FIG. 4: 3D pellets of fibroblasts reprogrammed to chondrocytes.

FIG. 5: Histological analysis of pellets from cells: A: control (A) and B: following treatment with small molecules [4-[(5,6,7,8-Tetrahydro-5,5,8,8-tetramethyl-2-naphthalenyl) carboxamido] benzoic acid (AM580), beta-estradiol, calcitriol, ciglitazone, kartogenin, lithium chloride, melatonin, and rhosin hydrochloride.

FIG. 6: Redifferentiation of culture expanded, dedifferentiated human articular chondrocytes using small molecule cocktail. Chondrocytes were isolated from human cartilage and expanded in 2D cultures through several passages (×6) to induce dedifferentiation. The expanded cells were incubated with or without small molecules for 2 weeks in 2D culture under normoxia and hyoxia. The small molecules provided to the cells are: [4-[(5,6,7,8-Tetrahydro-5,5,8,8-tetramethyl-2-naphthalenyl) carboxamido]benzoic acid (AM580), beta-estradiol, calcitriol, ciglitazone, kartogenin, lithium chloride, melatonin, and rhosin hydrochloride. SOX9 expression was quantified using qPCR against two housekeeping genes (eukaryotic translation elongation factor 2; EEF and β2-macroglobulin); n=1.

FIG. 7: Expression of the chondrocyte gene markers SMAD3, COL10A1, ACAN, COL2A1 and COL1A2 at various time points during the conversion of human dermal fibroblasts to chondrocytes.

FIG. 8: Immunofluorescence staining of cells during conversion of human fibroblasts (HDF) to chondrocytes. Cells were infected with lentivirus encoding the reprogramming factors (IRES-GFP) or control (GFP only). Column 1 shows nuclear staining by DAPI, Column 2 shows GFP staining, Column 3 shows staining for the chondrocyte marker Aggrecan and Column 4 is a phase image of the same field.

FIG. 9: Immunofluorescence staining of cells during conversion of human fibroblasts (HDF) to chondrocytes. Cells were infected with lentivirus encoding the transcription factors under the control of an inducible promoter or control (GFP only). Column 1 shows nuclear staining by DAPI, Column 2 shows GFP staining, Column 3 shows staining for the chondrocyte marker Aggrecan and Column 4 is a phase image of the same field.

FIG. 10: Immunofluorescence staining of cells during conversion of human fibroblasts (HDF) to chondrocytes. Cells were infected with lentivirus encoding the transcription factors under the control of an inducible promoter or control (GFP only). Column 1 shows nuclear staining by DAPI, Column 2 shows GFP staining, Column 3 shows staining for the chondrocyte marker RUNX2 and Column 4 is a phase image of the same field.

FIG. 11: Redifferentiation of culture expanded, dedifferentiated human articular chondrocytes using small molecules. qPCR data showing expression of SOX9 following incubation with small molecules. Negative control: DMSO (vehicle). Positive control: TGFβ. Small molecule cocktails: “All 8” (see Table 7); “E” (see Table 8) and “F” (see Table 9).

FIG. 12: Redifferentiation of culture expanded, dedifferentiated human articular chondrocytes using small molecules. qPCR data showing expression of SOX5 following incubation with small molecules. Negative control: DMSO (vehicle). Positive control: TGFβ. Small molecule cocktails: “All 8” (see Table 7); “E” (see Table 8) and “F” (see Table 9).

FIG. 13: Redifferentiation of culture expanded, dedifferentiated human articular chondrocytes using small molecules. qPCR data showing expression of Type II collagen following incubation with small molecules. Negative control: DMSO (vehicle). Positive control: TGFβ. Small molecule cocktails: “All 8” (see Table 7); “E” (see Table 8) and “F” (see Table 9).

FIG. 14: Redifferentiation of culture expanded, dedifferentiated human articular chondrocytes using small molecules. qPCR data showing expression of Type I Collagen (marker of de-differentiation) and Type X Collagen (marker of hypertrophic osteoarthritis) following incubation with small molecules. Negative control: DMSO (vehicle). Positive control: TGFβ. Small molecule cocktails: “All 8” (see Table 7); “E” (see Table 8) and “F” (see Table 9).

FIG. 15: Prevention of de-differentiation of freshly isolated primary chondrocytes using small molecules. qPCR data showing expression of SOX9 following incubation with small molecules. Small molecules prevented de-differentiation of chondrocytes. Negative control: DMSO (vehicle). Positive control: TGFβ. Small molecule cocktails: “All 8” (see Table 7); “E” (see Table 8) and “F” (see Table 9).

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa. As used herein, except where the context requires otherwise, the term “comprise” and variations of the term, such as “comprising”, “comprises” and “comprised”, are not intended to exclude further additives, components, integers or steps.

The process of reprogramming a cell alters the type of progeny a cell can produce and includes the distinct processes of forward programming and transdifferentiation. In some embodiments, forward programming of multipotent cells or pluripotent cells provides cells exhibiting at least one characteristic of a cell type having a more differentiated phenotype than the multipotent cell or pluripotent cell. In other embodiments, transdifferentiation of one somatic cell provides a cell exhibiting at least one characteristic of another somatic cell type.

The present invention provides compositions and methods for direct reprogramming or transdifferentiation of source cells to target cells, without the source cell becoming an induced pluripotent stem cell (iPS) intermediately prior to becoming a target cell. In comparison to iPS cell technology, transdifferentiation is highly efficient and poses a very low risk of teratoma formation for downstream applications. Moreover, transdifferentiation can be used in vivo for the direct conversion of one cell type into another, whereas iPS cell technology cannot.

The present invention is particularly directed towards the transdifferentiation of source cells into chondrocytes. These chondrocytes may have utility in a wide range of applications, including for autologous chondrocyte implantation in individuals who are suffering from any condition characterised by degenerative joint disease including where there is a need for replacement cartilage to be provided. Examples of conditions that may be treated in accordance with the methods of the present invention include osteoarthritis, rheumatoid arthritis, psoriatic arthritis, reactive arthritis and gonococcal arthritis.

The present invention also provides methods for “re-differentiation” of culture expanded chondrocytes which have become de-differentiated as a result of multiple culture passages. In addition, the present invention includes methods for preventing the de-differentiation of chondrocytes in expanded cell cultures. Thus, the methods have application in scenarios whereby primary chondrocytes are obtained from an individual and cultured in vitro. Prior art methods for culturing primary chondrocytes are known to result in the de-differentiation of the cells after several passages. However, the methods of the present invention overcome this limitation by providing methods and conditions for culturing chondrocytes so as to prevent de-differentiation or alternatively, to reverse the de-differentiation by “re-differentiating” the cells to chondrocytes.

Of course, the present invention also contemplates the scenario whereby cells are converted into chondrocytes in vivo, for example, by treating de-differentiated chondrocytes at the site of injury (for example in a damaged articular joint or other), according to the methods described herein, thereby stimulating the conversion of de-differentiated chondrocytes in vivo into re-differentiated, and therefore physiologically active chondrocytes.

As used herein, “transdifferentiation” refers to a method of reprogramming a somatic cell of one type (or having the characteristics of one type of somatic cell), such that the morphological and functional properties of the cell and converted into that of another cell type (without undergoing an intermediate pluripotent state or progenitor cell type). The term transdifferentiation may be used interchangeably with the terms “lineage reprogramming” or “cell reprogramming” or “cell conversion”. The term transdifferentiation necessarily includes circumstances where a cell has become de-differentiated such that it no longer possesses some or all of the characteristics of it differentiated cell type. In a particularly preferred application of the present information, de-differentiated chondrocytes can be re-programmed or “re-differentiated” into chondrocytes.

A source cell may be any cell type described herein, including a somatic cell or a diseased cell. The somatic cell may be an adult cell or a cell derived from an adult which displays one or more detectable characteristics of an adult or non-embryonic cell. The diseased cell may be a cell displaying one or more detectable characteristics of a disease or condition, for example the cell may be a de-differentiated chondrocyte obtained from an individual with a degenerative condition associated with loss of chondrogenic potential. Preferred source cells include fibroblasts (including dermal fibroblasts), embryonic stem cells, and de-differentiated chondrocytes.

As used herein, the term “somatic cell” refers to any cell forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. The somatic cells may be immortalized to provide an unlimited supply of cells, for example, by increasing the level of telomerase reverse transcriptase (TERT). For example, the level of TERT can be increased by increasing the transcription of TERT from the endogenous gene, or by introducing a transgene through any gene delivery method or system.

Unless otherwise indicated the methods for reprogramming source cells as described herein can be performed both in vivo and in vitro (where in vivo is practiced when somatic cells are present within a subject and therefore also includes in situ conversion, and where in vitro is practiced using isolated somatic cells maintained in culture).

An embryonic cell, such as an embryonic stem cell, may be a cell derived from an embryonic cell line and not directly derived from an embryo or fetus. Alternatively, the embryonic cell may be derived from an embryo or fetus however the cell is obtained or isolated without destruction of, or any negative influence on the development of, the embryo or fetus.

Differentiated somatic cells, including cells from a fetal, newborn, juvenile or adult primate, including human, individual, are suitable source cells in the methods of the invention. Suitable somatic cells include, but are not limited to, bone marrow cells, epithelial cells, endothelial cells, fibroblast cells, hematopoietic cells, keratinocytes, hepatic cells, intestinal cells, mesenchymal cells, myeloid precursor cells and spleen cells. Alternatively, the somatic cells can be cells that can themselves proliferate and differentiate into other types of cells, including blood stem cells, muscle/bone stem cells, brain stem cells and liver stem cells. Suitable somatic cells are receptive, or can be made receptive using methods generally known in the scientific literature, to uptake of transcription factors including genetic material encoding the transcription factors. Uptake-enhancing methods can vary depending on the cell type and expression system. Exemplary conditions used to prepare receptive somatic cells having suitable transduction efficiency are well-known by those of ordinary skill in the art. The starting somatic cells can have a doubling time of about twenty-four hours.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein, refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of target cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not target cells or their progeny as defined by the terms herein.

As used herein, reference to a “chondrocyte” is a reference to any cell that has the characteristics of a chondrocyte. A cell may be defined as having the characteristics of a chondrocyte based on one or more markers, including cell surface markers, gene expression levels or production of macromolecules. The characteristic may also be one or more morphological traits.

For example, in any embodiment of the present invention, a protein marker which is characteristic of a chondrocyte is SOX9, SOX5, SOX6, SMAD3, SMAD5, SMAD6, CD44, CD49, CD10, CD9, CD95, integrin α10β1, CD105, VEGF, COL2A1. In any embodiment of the present invention, the production of sulphated glycosaminoglycans (GAG), deposition of aggrecan, hyaluronic acid and production of type II collagen may be characteristic of a chondrocyte.

In any embodiment of the invention, a morphological trait which is characteristic of a chondrocyte may include the development of white extracellular matrix and the development of a cuboidal appearance.

The skilled person will also be familiar with means to distinguish the characteristics of source cells from those of chondrocytes (in other words, to test for the loss of source cell phenotype). For example, as provided in the examples herein, suitable source cells for the production of chondrocytes include dermal fibroblasts, embryonic stem cells and de-differentiated chondrocytes. The skilled person will be able to readily distinguish between characteristics of a dermal fibroblast and a chondrocyte, for example: the production of type I collagen is characteristic of a dermal fibroblast and is to be distinguished from the production of type II collagen, which is characteristic of a chondrocyte.

Furthermore, the skilled person will be able to distinguish between the characteristics of a de-differentiated chondrocyte and a (differentiated) chondrocyte. For example, de-differentiated chondrocytes are characterised by the gradual loss of differentiated chondrocyte marker gene expression (for example, reduced or lost expression of any one of SOX9, SMAD3, SMAD6, SMAD5, CD44, CD49, CD10, CD9, CD95, integrin α10β1, CD105, VEGF, SOX5/6, sulphated glycosaminoglycan GAG, aggrecan, hyaluronic acid and type II collagen). De-differentiated chondrocytes are also characterised by the increased expression of the fibroblastic gene, type I collagen.

A source cell is determined to be converted to a chondrocyte, or become a chondrocyte-like cell, by a method of the present invention when it displays at least one characteristic of a chondrocyte. For example, a human fibroblast will be identified as converted to a chondrocyte-like cell, when a fibroblast, following treatment according to the present invention, displays at least one characteristic of a chondrocyte. Typically, a cell will display 1, 2, 3, 4, 5, 6, 7, 8 or more characteristics of a chondrocyte. For example, a cell is identified or determined to be a chondrocyte-like cell when up-regulation of any one or more chondrocyte markers and/or change in cell morphology is detectable. Examples of chondrocyte markers include SOX9, COL2A1, SMAD3, SMAD5, SMAD6, CD44, CD49, CD10, CD9, CD95, Integrin α10β1, CD105 and the cell morphology is cuboidal appearance.

In any aspect of the invention, the chondrocyte characteristic may be determined by analysis of cell morphology, gene expression profiles, activity assay, protein expression profile, surface marker profile, or differentiation ability. Examples of characteristics or markers include those that are described herein and those known to the skilled person. Other examples of relevant markers include, for example for production of type II collagen, aggrecan and sulphated glycosaminoglycans (GAG) by the cell.

The transcription factors or other proteins that may be used to convert a dermal fibroblast to a cell that exhibits at least one characteristic of a chondrocyte are shown below in Table 1.

Any one or more of the transcription factors or proteins as shown in each row of Table 1 may be used to transdifferentiate a dermal fibroblast into a chondrocyte. For example, the protein expression or amount of any one, two, three, four, five, six, seven or all eight transcription factors as shown in each row may be used for the purposes of the present invention.

TABLE 1
Factors for reprogramming of dermal fibroblasts to chondrocytes

| TF1 | TF2 | TF3 | TF4 | TF5 | TF6 | TF7 | TF8 | % coverage |
|---|---|---|---|---|---|---|---|---|
| BARX1 | PITX1 | SMAD6 | FOXC1 | SIX2 | AHR | FOSB | JUNB | 97.75426 |
| PITX1 | SMAD6 | FOXC1 | SIX2 | AHR | PRRX2 | FOSB | JUNB | 97.75426 |
| SMAD6 | SIX1 | FOXC1 | SIX2 | AHR | PRRX2 | FOSB | JUNB | 97.44514 |
| GDF6 | TGFB3 | FOXC1 | SIX2 | AHR | PRRX2 | FOSB | JUNB | 97.44514 |
| RCAN1 | TGFB3 | FOXC1 | SIX2 | AHR | PRRX2 | FOSB | JUNB | 97.44514 |
| SIX1 | TGFB3 | FOXC1 | SIX2 | AHR | PRRX2 | FOSB | JUNB | 97.44514 |
| TGFB3 | FOXC1 | SIX2 | AHR | PRRX2 | FGF2 | FOSB | JUNB | 97.44514 |
| FOXC1 | SIX2 | HOXA11 | AHR | PRRX2 | FGF2 | FOSB | JUNB | 97.44514 |
| SIX2 | AHR | PRRX2 | FGF2 | FOSB | JUNB | VDR | RUNX1 | 98.06337 |
| HOXA11 | AHR | PRRX2 | FGF2 | FOSB | JUNB | VDR | HOXA7 | 98.37249 |
| AHR | PRRX2 | FGF2 | FOSB | JUNB | VDR | FOXQ1 | HOXA7 | 98.37249 |
| MEOX2 | PRRX2 | FGF2 | FOSB | JUNB | VDR | RUNX1 | HOXA7 | 98.37249 |
| DLX3 | PRRX2 | FGF2 | FOSB | JUNB | VDR | RUNX1 | HOXA7 | 98.37249 |
| ATOH8 | PRRX2 | FGF2 | FOSB | JUNB | VDR | RUNX1 | HOXA7 | 98.37249 |
| ID4 | PRRX2 | FGF2 | FOSB | JUNB | VDR | RUNX1 | HOXA7 | 98.37249 |
| AEBP1 | PRRX2 | FGF2 | FOSB | JUNB | VDR | RUNX1 | HOXA7 | 98.37249 |
| PRRX2 | FGF2 | FOSB | JUNB | VDR | FOXQ1 | RUNX1 | HOXA7 | 98.37249 |
| FHL2 | FGF2 | FOSB | JUNB | VDR | RUNX1 | HOXA7 | IRF1 | 97.44514 |
| EPAS1 | FGF2 | FOSB | JUNB | VDR | RUNX1 | HOXA7 | IRF1 | 97.44514 |
| NFATC4 | FGF2 | FOSB | JUNB | VDR | RUNX1 | HOXA7 | IRF1 | 97.44514 |
| FGF2 | FOSB | JUNB | VDR | FOXQ1 | RUNX1 | HOXA7 | IRF1 | 97.44514 |
| LMO4 | FOSB | JUNB | VDR | FOXQ1 | RUNX1 | HOXA7 | IRF1 | 97.44514 |
| NFKBIZ | FOSB | JUNB | VDR | FOXQ1 | RUNX1 | HOXA7 | IRF1 | 97.44514 |
| FOSB | JUNB | VEGFA | VDR | FOXQ1 | RUNX1 | HOXA7 | IRF1 | 97.44514 |
| JUNB | ICAM1 | VEGFA | VDR | FOXQ1 | RUNX1 | HOXA7 | IRF1 | 97.44514 |
| ICAM1 | VEGFA | VDR | FOXQ1 | RUNX1 | SMAD9 | HOXA7 | IRF1 | 93.73571 |
| VEGFA | DLX5 | VDR | FOXQ1 | RUNX1 | SMAD9 | HOXA7 | IRF1 | 93.73571 |
| DLX5 | PPARGC1A | VDR | FOXQ1 | RUNX1 | SMAD9 | HOXA7 | IRF1 | 93.58116 |
| PPARGC1A | INHBA | VDR | FOXQ1 | RUNX1 | SMAD9 | HOXA7 | IRF1 | 93.58116 |
| WWTR1 | INHBA | VDR | FOXQ1 | RUNX1 | SMAD9 | HOXA7 | IRF1 | 93.58116 |
| PKNOX2 | INHBA | VDR | FOXQ1 | RUNX1 | SMAD9 | HOXA7 | IRF1 | 93.58116 |
| MYOCD | INHBA | VDR | FOXQ1 | RUNX1 | SMAD9 | HOXA7 | IRF1 | 93.58116 |
| INHBA | VDR | FOXQ1 | RUNX1 | SMAD9 | IL6 | HOXA7 | IRF1 | 93.58116 |
| VDR | FOXQ1 | RUNX1 | SMAD9 | BMP4 | IL6 | HOXA7 | IRF1 | 93.58116 |
| CUX1 | FOXQ1 | RUNX1 | SMAD9 | BMP4 | HOXA7 | EBF1 | IRF1 | 92.80836 |
| RIPK3 | FOXQ1 | RUNX1 | SMAD9 | BMP4 | HOXA7 | EBF1 | IRF1 | 92.80836 |
| TSC22D1 | FOXQ1 | RUNX1 | SMAD9 | BMP4 | HOXA7 | EBF1 | IRF1 | 92.6538 |
| FOXQ1 | RUNX1 | SMAD9 | BMP4 | IL6 | HOXA7 | EBF1 | IRF1 | 92.6538 |
| RUNX1 | SMAD9 | BMP4 | HMOX1 | IL6 | HOXA7 | EBF1 | IRF1 | 90.02629 |
| IGF2 | SMAD9 | BMP4 | IL6 | HOXA7 | ID1 | EBF1 | IRF1 | 89.40806 |
| SMAD9 | BMP4 | HMOX1 | IL6 | HOXA7 | ID1 | EBF1 | IRF1 | 89.40806 |
| VGLL4 | BMP4 | HMOX1 | IL6 | HOXA7 | ID1 | EBF1 | IRF1 | 85.69863 |
| TRPS1 | BMP4 | HMOX1 | IL6 | HOXA7 | ID1 | EBF1 | IRF1 | 85.69863 |
| SOX9 | PPAR γ | BMP-2 | PITX1 | TGFβ3 | VDR | RUNX1 | — | 85.65 |

As used herein, percentage coverage (% coverage) refers to the percentage of genes that are directly regulated by the listed transcription factors and for which expression is predicted to be altered between the source cell and the target cell type. For example, the transcription factors shown in row 1 of Table 1 directly regulate the expression of 97.75% of those genes whose expression is being targeted in order to convert the source cell to the target cell.

The inventors have also found, that in addition to the above preferred groupings of transcription factors, there are some transcription factors or proteins which can be readily substituted for others. For example, in the context of the preferred group of transcription factors as shown in row 1 of Table 1, BARX1, PITX1, SMAD6, FOXC1, SIX2, AHR, FOSB and JUNB, the inventors have found that any of these transcription factors can be replaced with the transcription factor PPRX2. In other words, if it is not possible to increase the protein expression or amount of any of the given factors BARX1, PITX1, SMAD6, FOXC1, SIX2, AHR, FOSB and JUNB, (for example, if there is no nucleic acid construct available for increasing expression directly, or there is no small molecule which directly targets the transcription factor for stimulating increased expression), then it is possible to seek to increase the expression of PPRX2 instead. Put in other words, the transcription factor PPRX2 can substitute for any of BARX1, PITX1, SMAD6, FOXC1, SIX2, AHR, FOSB and JUNB, when seeking to transdifferentiate a dermal fibroblast to a chondrocyte, according to the present methods.

The transcription factors that may be used to convert an embryonic stem cell to a cell that exhibits at least one characteristic of a chondrocyte are shown below in Table 2.

Any one or more of the transcription factors as shown in each row of Table 2 may be used to convert an embryonic stem cell into a chondrocyte. For example, the protein expression or amount of any one, two, three, four, five, six, seven or all eight transcription factors as shown in each row may be used for the purposes of the present invention

TABLE 2
Transcription factors for reprogramming embryonic stem cells to chondrocytes

| TF 1 | TF2 | TF3 | TF4 | TF5 | TF6 | TF7 | TF8 | % Coverage |
|---|---|---|---|---|---|---|---|---|
| BARX1 | PITX1 | SMAD6 | NFKB1 | FOXC1 | AHR | SIX2 | JUNB | 99.41713 |
| PITX1 | SMAD6 | NFKB1 | HOXA11 | FOXC1 | AHR | SIX2 | JUNB | 99.41713 |
| SMAD6 | NFKB1 | HOXA11 | FOXC1 | AHR | SIX2 | PRRX2 | JUNB | 99.32402 |
| NFKB1 | ZEB2 | TGFB3 | FOXC1 | AHR | SIX2 | PRRX2 | JUNB | 99.32402 |
| RCAN1 | TGFB3 | FOXC1 | AHR | SIX2 | PRRX2 | IRF1 | JUNB | 99.04469 |
| GDF6 | TGFB3 | FOXC1 | AHR | SIX2 | PRRX2 | IRF1 | JUNB | 99.04469 |
| HOXC10 | TGFB3 | FOXC1 | AHR | SIX2 | PRRX2 | IRF1 | JUNB | 99.04469 |
| HOXA11 | TGFB3 | FOXC1 | AHR | SIX2 | PRRX2 | IRF1 | JUNB | 99.04469 |
| SIX1 | TGFB3 | FOXC1 | AHR | SIX2 | PRRX2 | IRF1 | JUNB | 99.04469 |
| ZEB2 | TGFB3 | FOXC1 | AHR | SIX2 | PRRX2 | IRF1 | JUNB | 99.04469 |
| TGFB3 | FOXC1 | AHR | SIX2 | PRRX2 | IRF1 | JUNB | HOXA7 | 99.88268 |
| FOXC1 | AHR | SIX2 | PRRX2 | IRF1 | JUNB | HOXA9 | HOXA7 | 99.88268 |
| AHR | SIX2 | PRRX2 | IRF1 | JUNB | EBF1 | NFIX | VDR | 98.76537 |
| SIX2 | PRRX2 | IRF1 | JUNB | EBF1 | NFIX | VDR | HOXA7 | 99.23091 |
| MEOX2 | PRRX2 | IRF1 | JUNB | EBF1 | NFIX | VDR | HOXA7 | 98.95158 |
| AEBP1 | PRRX2 | IRF1 | JUNB | EBF1 | NFIX | VDR | HOXA7 | 98.95158 |
| DLX3 | PRRX2 | IRF1 | JUNB | EBF1 | NFIX | VDR | HOXA7 | 98.95158 |
| PRRX2 | IRF1 | JUNB | EBF1 | HOXA9 | NFIX | VDR | HOXA7 | 98.95158 |
| IRF1 | JUNB | EBF1 | HOXA9 | NFIX | PRRX1 | VDR | HOXA7 | 98.95158 |
| JUNB | EBF1 | HOXA9 | NFIX | PRRX1 | VDR | HOXA7 | NR2F2 | 99.23091 |
| ATOH8 | EBF1 | HOXA9 | NFIX | PRRX1 | FOSB | VDR | HOXA7 | 96.81006 |
| FHL2 | EBF1 | HOXA9 | NFIX | PRRX1 | FOSB | VDR | HOXA7 | 96.81006 |
| EBF1 | HOXA9 | NFIX | PRRX1 | FOSB | VDR | HOXA7 | FOXQ1 | 97.64805 |
| TWIST1 | HOXA9 | NFIX | PRRX1 | FOSB | VDR | HOXA7 | FOXQ1 | 97.46183 |
| NFATC4 | HOXA9 | NFIX | PRRX1 | FOSB | VDR | HOXA7 | FOXQ1 | 97.46183 |
| HOXA9 | NFIX | PRRX1 | FOSB | VDR | HOXA7 | FOXQ1 | RUNX1 | 98.11360 |
| NFIX | PRRX1 | FOSB | VDR | HOXA7 | FOXQ1 | RUNX1 | NR2F2 | 98.76537 |
| PRRX1 | EPAS1 | FOSB | VDR | HOXA7 | FOXQ1 | RUNX1 | NR2F2 | 98.76537 |
| TBX2 | FOSB | VEGFA | VDR | HOXA7 | RUNX1 | NR2F2 | SMAD9 | 97.27561 |
| FOSB | VEGFA | VDR | HOXA7 | FOXQ1 | RUNX1 | NR2F2 | SMAD9 | 97.27561 |
| EPAS1 | VEGFA | VDR | HOXA7 | FOXQ1 | RUNX1 | NR2F2 | SMAD9 | 96.15829 |
| VEGFA | VDR | HOXA7 | FOXQ1 | RUNX1 | NR2F2 | SMAD9 | JUN | 97.46183 |
| PPARGC1A | INHBA | VDR | HOXA7 | FOXQ1 | RUNX1 | NR2F2 | SMAD9 | 95.87896 |
| INHBA | VDR | HOXA7 | FOXQ1 | RUNX1 | NR2F2 | SMAD9 | JUN | 97.27561 |
| RARG | VDR | HOXA7 | FOXQ1 | RUNX1 | NR2F2 | SMAD9 | JUN | 97.27561 |
| LMO4 | VDR | HOXA7 | FOXQ1 | RUNX1 | NR2F2 | SMAD9 | JUN | 97.27561 |
| MYBL1 | VDR | HOXA7 | FOXQ1 | RUNX1 | NR2F2 | SMAD9 | JUN | 97.27561 |
| NFKBIZ | VDR | HOXA7 | FOXQ1 | RUNX1 | NR2F2 | SMAD9 | JUN | 97.27561 |
| ENPP2 | VDR | HOXA7 | FOXQ1 | RUNX1 | NR2F2 | SMAD9 | JUN | 97.27561 |
| WWTR1 | VDR | HOXA7 | FOXQ1 | RUNX1 | NR2F2 | SMAD9 | JUN | 97.27561 |
| PKNOX2 | VDR | HOXA7 | FOXQ1 | RUNX1 | NR2F2 | SMAD9 | JUN | 97.27561 |
| MYOCD | VDR | HOXA7 | FOXQ1 | RUNX1 | NR2F2 | SMAD9 | JUN | 97.27561 |
| VDR | HOXA7 | FOXQ1 | RUNX1 | NR2F2 | SMAD9 | JUN | ELF4 | 98.20671 |

The inventors have also found, that in addition to the above preferred groupings of transcription factors as shown in Table 2, there are some transcription factors which can be readily substituted for others. For example, in the context of the preferred group of transcription factors as shown in row 1 of Table 2, BARX1, PITX1, SMAD6, NFKB, FOXC1, AHR, SIX2 and JUNB, the inventors have found that any of the transcription factors PITX1, FOXC1, SIX2 and AHR can be replaced with the transcription PPRX2. In other words, if it is not possible to increase the protein expression or amount of any of the given transcription factors PITX1, FOXC1, SIX2 and AHR (for example, if there is no nucleic acid construct available for increasing expression directly, or there is no small molecule which directly targets the transcription factor for stimulating increased expression), then it is possible to seek to increase the expression of PPRX2 instead. Put in other words, the transcription factor PPRX2 can substitute for any of PITX1, FOXC1, SIX2 and AHR when seeking to transdifferentiate an embryonic stem cell to a chondrocyte, according to the present methods.

Still further, the inventors have found that in the context of the preferred group of transcription factors as shown in row 1 of Table 2, the transcription factor BARX1 can be replaced with HOXA11. Further the transcription factor SMAD6 can be replaced with TGFβ3, NFkB can be replaced with IRF1 and JUNB can be replaced with FOSB. In other words, if it is not possible to increase the protein expression or amount of any of the given transcription factors BARX1, SMAD6, NFkB and JUNB (for example, if there is no nucleic acid construct available for increasing expression directly, or there is no small molecule which directly targets the transcription factor for stimulating increased expression), then it is possible to seek to increase the expression of HOXA11, TGFβ3, IRF1 or JUNB, respectively, instead.

The transcription factors that may be used to convert a de-differentiated chondrocyte to a cell that exhibits at least one characteristic of a chondrocyte are shown below in Table 3.

Any one or more of the transcription factors as shown in each row of Table 2 may be used to convert a de-differentiated chondrocyte into a chondrocyte. For example, the protein expression or amount of any one, two, three, four, five, six, seven or all eight transcription factors as shown in each row may be used for the purposes of the present invention

TABLE 3
Transcription factors for reprogramming de-differentiated chondrocytes to chondrocytes

| TF1 | TF2 | TF3 | TF4 | TF5 | TF6 | TF7 | TF8 | % coverage |
|---|---|---|---|---|---|---|---|---|
| SOX9 | SOX5 | VEGFA | RUNX1 | VDR | NR2F1 | FOSB | PRRX1 | 98.01492 |
| IL11 | SOX5 | VEGFA | RUNX1 | VDR | NR2F1 | FOSB | PRRX1 | 95.84396 |
| SOX5 | VEGFA | RUNX1 | VDR | NR2F1 | FOSB | HOXA7 | PRRX1 | 95.84396 |
| VEGFA | RUNX1 | VDR | TCF4 | NR2F1 | FOSB | HOXA7 | PRRX1 | 95.84396 |
| BMP2 | RUNX1 | VDR | TCF4 | NR2F1 | FOSB | HOXA7 | PRRX1 | 95.70828 |
| AEBP1 | RUNX1 | VDR | TCF4 | NR2F1 | FOSB | HOXA7 | PRRX1 | 95.70828 |
| S100A1 | RUNX1 | VDR | TCF4 | NR2F1 | FOSB | HOXA7 | PRRX1 | 95.70828 |
| MDFI | RUNX1 | VDR | TCF4 | NR2F1 | FOSB | HOXA7 | PRRX1 | 95.70828 |
| TSC22D1 | RUNX1 | VDR | TCF4 | NR2F1 | FOSB | HOXA7 | PRRX1 | 95.70828 |
| RUNX1 | VDR | TGFB3 | TCF4 | NR2F1 | FOSB | HOXA7 | PRRX1 | 95.70828 |
| ERG | VDR | TGFB3 | TCF4 | NR2F1 | FOSB | HOXA7 | PRRX1 | 94.75848 |
| TRPS1 | VDR | TGFB3 | TCF4 | NR2F1 | FOSB | HOXA7 | PRRX1 | 94.75848 |
| GAS7 | VDR | TGFB3 | TCF4 | NR2F1 | FOSB | HOXA7 | PRRX1 | 94.75848 |
| WWTR1 | VDR | TGFB3 | TCF4 | NR2F1 | FOSB | HOXA7 | PRRX1 | 94.62280 |
| PKNOX2 | VDR | TGFB3 | TCF4 | NR2F1 | FOSB | HOXA7 | PRRX1 | 94.62280 |
| VDR | TGFB3 | DLX5 | TCF4 | NR2F1 | FOSB | HOXA7 | PRRX1 | 94.62280 |
| INHBA | TGFB3 | DLX5 | TCF4 | NR2F1 | FOSB | HOXA7 | PRRX1 | 94.21574 |
| ARHGAP22 | TGFB3 | DLX5 | TCF4 | NR2F1 | FOSB | HOXA7 | PRRX1 | 94.21574 |
| TGFB3 | DLX5 | CUX1 | TCF4 | NR2F1 | FOSB | HOXA7 | PRRX1 | 94.21574 |
| DLX5 | CUX1 | TCF4 | ATF3 | NR2F1 | FOSB | HOXA7 | PRRX1 | 94.21574 |
| ZNF70 | CUX1 | TCF4 | ATF3 | NR2F1 | FOSB | HOXA7 | PRRX1 | 94.21574 |
| VGLL4 | CUX1 | TCF4 | ATF3 | NR2F1 | FOSB | HOXA7 | PRRX1 | 94.21574 |
| RFX8 | CUX1 | TCF4 | ATF3 | NR2F1 | FOSB | HOXA7 | PRRX1 | 94.21574 |
| CUX1 | TCF4 | ATF3 | NR2F1 | HMOX1 | FOSB | HOXA7 | PRRX1 | 94.21574 |
| TCF4 | ATF3 | NR2F1 | HMOX1 | BRD8 | FOSB | HOXA7 | PRRX1 | 94.21574 |
| ATF3 | NR2F1 | HMOX1 | BRD8 | JMJD1C | FOSB | HOXA7 | PRRX1 | 93.53732 |
| NR2F1 | HMOX1 | BRD8 | ZNF267 | JMJD1C | FOSB | HOXA7 | PRRX1 | 93.53732 |
| HMOX1 | BRD8 | ZNF267 | JMJD1C | FOSB | HOXA7 | NR2F2 | PRRX1 | 93.53732 |
| BRD8 | ZNF267 | JMJD1C | FOSB | HOXA7 | NR2F2 | PRRX1 | N/A | 93.53732 |
| ZNF267 | JMJD1C | FOSB | HOXA7 | NR2F2 | PRRX1 | N/A | N/A | 93.53732 |
| PHTF2 | JMJD1C | FOSB | HOXA7 | NR2F2 | PRRX1 | N/A | N/A | 93.53732 |
| JMJD1C | FOSB | HOXA7 | NR2F2 | PRRX1 | N/A | N/A | N/A | 93.53732 |
| FOSB | HOXA7 | NR2F2 | PRRX1 | N/A | N/A | N/A | N/A | 93.53732 |
| CYTL1 | HOXA7 | MXD1 | NFKBIA | NR2F2 | TSC22D3 | PRRX1 | IGF1 | 91.36636 |
| HOXA7 | MXD1 | NFKBIA | NR2F2 | TSC22D3 | PRRX1 | IGF1 | SP110 | 91.50204 |
| MXD1 | NFKBIA | NR2F2 | TSC22D3 | PRRX1 | IGF1 | SP110 | PIM1 | 78.6119707 |
| NFKBIA | NR2F2 | TSC22D3 | PRRX1 | IGF1 | SP110 | PIM1 | N/A | 78.4762856 |
| NR2F2 | TSC22D3 | PRRX1 | IGF1 | SP110 | PIM1 | N/A | N/A | 77.2551204 |
| LBH | TSC22D3 | PRRX1 | IGF1 | SP110 | PIM1 | N/A | N/A | 66.8073734 |
| TSC22D3 | PRRX1 | IGF1 | SP110 | PIM1 | N/A | N/A | N/A | 66.8073734 |
| CREB3L2 | PRRX1 | IGF1 | SP110 | PIM1 | N/A | N/A | N/A | 66.5360033 |
| PRRX1 | IGF1 | SP110 | PIM1 | N/A | N/A | N/A | N/A | 66.5360033 |

The inventors have also found that in addition to the above preferred groupings of transcription factors as shown in Table 3, there are some transcription factors which can be readily substituted for others. For example, in the context of the preferred group of transcription factors as shown in row 1 of Table 3, SOX9, SOX5, VEGFA, RUNX1, VDR, NR2F1, FOSB and PRRX1, the inventors have found that the transcription factor SOX9 can be replaced with the protein IL11. In other words, if it is not possible to increase the protein expression or amount of the transcription factor SOX9 (for example, if there is no nucleic acid construct available for increasing expression directly, or there is no small molecule which directly targets the transcription factor for stimulating increased expression), then it is possible to seek to increase the expression of IL11 instead. Put in other words, IL11 can substitute for SOX9 when seeking to transdifferentiate a de-differentiated chondrocyte to a (re-differentiated) chondrocyte, according to the present methods.

Still further, the inventors have found that in the context of the preferred group of transcription factors as shown in row 1 of Table 3, the transcription factors SOX5, RUNX1 and VDR can be replaced with TCF4. Further the transcription factors VEGFA, NR2F1, FOSB and PRRX1 can be replaced with HOXA7. NR2F1 can be replaced with either HOXA7 or NR2F2.

The transcription factors and proteins referred to herein are referred to by the HUGO Gene Nomenclature Committee (HGNC) Symbol. Table 4 provides exemplary Ensemble Gene ID and Uniprot IDs for the factors recited herein. The nucleotide sequences are derived from the Ensembl database (Flicek et al. (2014). Nucleic Acids Research Volume 42, Issue D1. Pp. D749-D755) version 83. Also contemplated for use in the invention is any homolog, ortholog or paralog of a factor referred to herein.

The skilled person will appreciate that this information may be used in performing the methods of the present invention, for example, for the purposes of providing increased amounts of transcription factors in source cells, or providing nucleic acids or the like for recombinantly expressing a transcription factor in a source cell.

TABLE 4
Accession numbers identifying nucleotide sequences and amino acid
sequences of transcription factors and proteins referred to herein.

| Transcription factor | | |
|---|---|---|
| Associated Gene Name | Ensembl Gene ID | Uniprot ID |
| AEBP1 | ENSG00000106624 | Q8IUX7 |
| AHR | ENSG00000106546 | P35869 |
| ANKRD1 | ENSG00000148677 | Q15327 |
| ARNT2 | ENSG00000172379 | Q9HBZ2 |
| ATOH8 | ENSG00000168874 | Q96SQ7 |
| BACH2 | ENSG00000112182 | Q9BYV9 |
| BARX1 | ENSG00000131668 | Q9HBU1 |
| BMP4 | ENSG00000125378 | P12644 |
| BRD8 | ENSG00000112983 | Q9H0E9 |
| CDH1 | ENSG00000039068 | P12830 |
| CDH1 | ENSG00000039068 | P12830 |
| CEBPB | ENSG00000172216 | P17676 |
| CREB3 | ENSG00000107175 | O43889 |
| CUX1 | ENSG00000257923 | Q13948 |
| DBP | ENSG00000105516 | Q10586 |
| DLX3 | ENSG00000064195 | O60479 |
| DLX5 | ENSG00000105880 | P56178 |
| E2F1 | ENSG00000101412 | Q01094 |
| E2F5 | ENSG00000133740 | Q15329 |
| EBF1 | ENSG00000164330 | Q9UH73 |
| ELF4 | ENSG00000102034 | Q99607 |
| ENPP2 | ENSG00000136960 | Q13822 |
| EPAS1 | ENSG00000116016 | Q99814 |
| ESRRA | ENSG00000173153 | P11474 |
| FGF2 | ENSG00000138685 | P09038 |
| FHL2 | ENSG00000115641 | Q14192 |
| FOS | ENSG00000170345 | P01100 |
| FOSB | ENSG00000125740 | P53539 |
| FOSL1 | ENSG00000175592 | P15407 |
| FOSL2 | ENSG00000075426 | P15408 |
| FOSL2 | ENSG00000075426 | P15408 |
| FOXA2 | ENSG00000125798 | Q9Y261 |
| FOXC1 | ENSG00000054598 | Q12948 |
| FOXC2 | ENSG00000176692 | Q99958 |
| FOXD1 | ENSG00000251493 | Q16676 |
| FOXQ1 | ENSG00000164379 | Q9C009 |
| GATA1 | ENSG00000102145 | P15976 |
| GATA6 | ENSG00000141448 | Q92908 |
| GDF6 | ENSG00000156466 | Q6KF10 |
| GFI1 | ENSG00000162676 | Q99684 |
| GFI1B | ENSG00000165702 | Q5VTD9 |
| HES1 | ENSG00000114315 | Q14469 |
| HIC1 | ENSG00000177374 | Q14526 |
| HMGB2 | ENSG00000164104 | P26583 |
| HMOX1 | ENSG00000100292 | P09601 |
| HOXA7 | ENSG00000122592 | P31268 |
| HOXA9 | ENSG00000078399 | P31269 |
| HOXA11 | ENSG00000005073 | P31270 |
| HOXB7 | ENSG00000260027 | P09629 |
| HOXC10 | ENSG00000180818 | Q9NYD6 |
| HR | ENSG00000168453 | O43593 |
| ICAM1 | ENSG00000090339 | P05362 |
| ID1 | ENSG00000125968 | P41134 |
| ID4 | ENSG00000172201 | P47928 |
| IGF1 | ENSG00000017427 | P05019 |
| IGF2 | ENSG00000167244 | P01344 |
| IL1B | ENSG00000125538 | P01584 |
| IL6 | ENSG00000136244 | P05231 |
| IL11 | ENSG00000095752 | P20809 |
| INHBA | ENSG00000122641 | P08476 |
| IRF1 | ENSG00000125347 | P10914 |
| IRX5 | ENSG00000176842 | P78411 |
| JMJD1C | ENSG00000171988 | Q15652 |
| JUN | ENSG00000177606 | P05412 |
| JUNB | ENSG00000171223 | P17275 |
| KLF1 | ENSG00000105610 | Q13351 |
| LEF1 | ENSG00000138795 | Q9UJU2 |
| LIF | ENSG00000128342 | P15018 |
| LMO3 | ENSG00000048540 | Q8TAP4 |
| LMO4 | ENSG00000143013 | P61968 |
| MAFB | ENSG00000204103 | Q9Y5Q3 |
| MEIS1 | ENSG00000143995 | O00470 |
| MEOX2 | ENSG00000106511 | P50222 |
| MSX1 | ENSG00000163132 | P28360 |
| MXD4 | ENSG00000123933 | Q14582 |
| MYB | ENSG00000118513 | P10242 |
| MYBL1 | ENSG00000185697 | P10243 |
| MYBL2 | ENSG00000101057 | P10244 |
| MYC | ENSG00000136997 | P01106 |
| MYOD1 | ENSG00000129152 | P15172 |
| MYOCD | ENSG00000141052 | Q8IZQ8 |
| MYOG | ENSG00000122180 | P15173 |
| NFATC2 | ENSG00000101096 | Q13469 |
| NFATC4 | ENSG00000100968 | Q14934 |
| NFE2 | ENSG00000123405 | Q16621 |
| NFIX | ENSG00000008441 | Q14938 |
| NFKB1 | ENSG00000109320 | P19838 |
| NFKBIA | ENSG00000100906 | P25963 |
| NFKBIZ | ENSG00000144802 | Q9BYH8 |
| NKX2-1 | ENSG00000136352 | P43699 |
| NOTCH1 | ENSG00000148400 | P46531 |
| NOTCH3 | ENSG00000074181 | Q9UM47 |
| NR2F1 | ENSG00000175745 | P10589 |
| (COUP-TF1) | | |
| NR2F2 | ENSG00000185551 | P24468 |
| NR3C1 | ENSG00000113580 | P04150 |
| OTX1 | ENSG00000115507 | P32242 |
| PAX6 | ENSG00000007372 | P26367 |
| PBX1 | ENSG00000185630 | P40424 |
| PITX1 | ENSG00000069011 | P78337 |
| PITX3 | ENSG00000107859 | O75364 |
| PKNOX2 | ENSG00000165495 | Q96KN3 |
| PPARGCIA | ENSG00000109819 | Q9UBK2 |
| PKNOX2 | ENSG00000165495 | Q96KN3 |
| POU3F2 | ENSG00000184486 | P20265 |
| PRRX1 | ENSG00000116132 | P54821 |
| PRRX2 | ENSG00000167157 | Q99811 |
| RARB | ENSG00000077092 | P10826 |
| RARG | ENSG00000172819 | P13631 |
| RCAN1 | ENSG00000159200 | P53805 |
| REL | ENSG00000162924 | Q04864 |
| RIPK3 | ENSG00000129465 | Q9Y572 |
| RORA | ENSG00000069667 | P35398 |
| RUNX1 | ENSG00000159216 | Q01196 |
| RUNX1T1 | ENSG00000079102 | Q06455 |
| RUNX2 | ENSG00000124813 | Q13950 |
| RUNX3 | ENSG00000020633 | Q13761 |
| SIX1 | ENSG00000126778 | Q15475 |
| SIX2 | ENSG00000170577 | Q9NPC8 |
| SIX5 | ENSG00000177045 | Q8N196 |
| SMAD1 | ENSG00000170365 | Q15797 |
| SMAD6 | ENSG00000137834 | O43541 |
| SMAD7 | ENSG00000101665 | O15105 |
| SMAD9 | ENSG00000120693 | O15198 |
| SNAI2 | ENSG00000019549 | O43623 |
| SOX17 | ENSG00000164736 | Q9H6I2 |
| SOX2 | ENSG00000181449 | P48431 |
| SOX5 | ENSG00000134532 | P35711 |
| SOX8 | ENSG00000005513 | P57073 |
| SOX9 | ENSG00000125398 | P48436 |
| SREBF1 | ENSG00000072310 | P36956 |
| TAL1 | ENSG00000162367 | P17542 |
| TCF7L1 | ENSG00000152284 | Q9HCS4 |
| TFAP2A | ENSG00000137203 | P05549 |
| TGFB3 | ENSG00000119699 | P10600 |
| TP63 | ENSG00000073282 | Q9H3D4 |
| TRPS1 | ENSG00000104447 | Q9UHF7 |
| TSC22D1 | ENSG00000102804 | Q15714 |
| TWIST1 | ENSG00000122691 | Q15672 |
| VDR | ENSG00000111424 | P11473 |
| VEGFA | ENSG00000112715 | P15692 |
| VGLL4 | ENSG00000144560 | Q14135 |
| WWTR1 | ENSG00000018408 | Q9GZV5 |
| ZEB2 | ENSG00000169554 | O60315 |
| ZFP42 | ENSG00000179059 | Q96MM3 |
| ZIC1 | ENSG00000152977 | Q15915 |

The term a “variant” in referring to a polypeptide that is at least 70%, 80%, 85%, 90%, 95%, 98%, or 99% identical to the full length polypeptide. The present invention contemplates the use of variants of the transcription factors described herein, including variants of the transcription factors listed in tables 1 and 2 and the sequences listed in Table 3. The variant could be a fragment of full length polypeptide or a naturally occurring splice variant. The variant could be a polypeptide at least 70%, 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof has a functional activity of interest such as the ability to promote conversion of a source cell type to a target cell type. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein. One of skill in the art will be aware of, or will readily be able to ascertain, whether a particular polypeptide variant, fragment, or derivative is functional using assays known in the art. For example, the ability of a variant of a transcription factor to convert a source cell to a target cell type can be assessed using the assays as disclose herein in the Examples. Other convenient assays include measuring the ability to activate transcription of a reporter construct containing a transcription factor binding site operably linked to a nucleic acid sequence encoding a detectable marker such as luciferase. In certain embodiments of the invention a functional variant or fragment has at least 50%, 60%, 70%, 80%, 90%, 95% or more of the activity of the full length wild type polypeptide.

The term “increasing the amount of” with respect to increasing an amount of a transcription factor, refers to increasing the quantity of the transcription factor in a cell of interest (e.g., a source cell such as a fibroblast cell). In some embodiments, the amount of transcription factor is “increased” in a cell of interest (e.g., a cell into which an expression cassette directing expression of a polynucleotide encoding one or more transcription factors has been introduced) when the quantity of transcription factor is at least 10%, 20%, 30%, 40%, 50%, 60%, 70%, 80%, 90%, or more relative to a control (e.g., a fibroblast into which none of said expression cassettes have been introduced). However, any method of increasing an amount of a transcription factor is contemplated including any method that increases the amount, rate or efficiency of transcription, translation, stability or activity of a transcription factor (or the pre-mRNA or mRNA encoding it). In addition, down-regulation or interference of a negative regulator of transcription expression, increasing efficiency of existing translation (e.g. SINEUP) are also considered.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

In a particularly preferred embodiment of the present invention, the agent is a small molecule.

Where a small molecule is used for activating, for increasing the amount of a transcription factor or for increasing expression of a gene encoding a transcription factor, the small molecule may be selected from the group including:
[4-[(5,6,7,8-Tetrahydro-5,5,8,8-tetramethyl-2-naphthalenyl) carboxamido]benzoic acid (AM580),
beta-estradiol,
calcitriol,
ciglitazone,
kartogenin,
Lithium chloride,
melatonin,
rhosin hydrochloride,
leucovorin calcium (folate),
forskolin, and
retinoic acid (All trans or 9-cis).

As used herein, AM580, refers to 4-[(5,6,7,8-Tetrahydro-5,5,8,8-tetramethyl-2-naphthalenyl)carboxamido] benzoic acid (CAS number 102121-60-8) and is an analog of retinoic acid that acts as a selective RARα agonist. AM580 is a potent stimulator of the transcription factor SOX9, which plays a pivotal role during chondrocyte differentiation. AM580 can be purchased from a number of commercial suppliers, including from Tocris under the catalogue number 0760.

It will be understood that any analog of retinoic acid may have utility in the methods of the present invention, including for increasing/stimulating expression of SOX9. For example, in addition to utilising the retinoic analog AM580, the small molecule may be (All trans)-retinoic acid or 9-cis retinoic acid. (All-trans)-retinoic acid (CAS no: 302-79-4) is also known as Tretinoin, or ATRA and can be purchased from a number of commercial suppliers, including from Sigma under the catalogue number R2625. 9-cis-retinoic acid, also known as Alitretinoin (CAS no: 5300-03-8) can be purchased from a number of commercial suppliers, including from Sigma under the catalogue number R4643.

In addition to increasing the amount of SOX9, retinoic acid (and analogs thereof such as AM580) can be used to activate/increase the amount of NR2F1, RUNX1, SOX5, and HOXA7.

As used herein, beta-estradiol (also spelt beta-oestradiol or β-estradiol) refers to the endogenous oestrogen receptor agonist, which is also referred to as 8R, 9S, 13S, 14S, 17S)-13-methyl-6,7,8,9,11,12,14,15,16,17-decahydrocyclopenta[α]phenanthrene-3,17-diol, Estra-1,3,5(10)-triene-3,17β-diol or 17β-estradiol. Beta-estradiol may also be referred to by its CAS number 50-28-2. Beta-estradiol can stimulate the expression of the transcription factor PITX1, which is critical for the differentiation and maturation of chondrogenic cells. Loss or inactivation of PITX1 leads to loss of normal hind leg development, and the prevention of adequate cartilage development. Beta-estradiol can be purchased from a number of commercial suppliers, including from Tocris under the catalogue number 2824.

As used herein, calcitriol refers to 1,25-dihydroxycholecalciferol or 1,25-dihydroxyvitamin D3 (CAS number 32222-06-3). Calcitriol is the hormonally active metabolite of vitamin D and is an activator of the vitamin D receptor (VDR). VDR is a nuclear receptor which is a regulator of osteoclastogenesis within chondrocytes and has an effect on hypertrophic differentiation. Calcitriol may also be used to indirectly stimulate expression of the transcription factor VEGF via its stimulation of VDR. In addition, calcitriol can be used to activate/increase the amount of JUNB. Calcitriol can be purchased from a number of commercial suppliers, including from Tocris under the catalogue number 2551.

As used herein, ciglitazone refers to a selective agonist for peroxisome proliferator-activated receptor γ (PPAR γ). Ciglitazone is a thiazolidinedione having CAS number 74772-77-3 and can be purchased from a number of commercial suppliers, including from Tocris under the catalog number 1307. Ciglitazone is a selective agonist of the peroxisome proliferator-activated receptor γ (PPAR γ), which in turn may also stimulate expression of VEGFA. Stimulation of the vascular system through activation of PPAR γ and consequently VEGFA within cartilage may also lead to the promotion of healing with fully functional tissue.

As used herein, kartogenin refers to 2-[(Biphenyl-4-yl)carbamoyl]benzoic acid, N-biphenul-4-yl-pthalmic acid or 4′-Phenyl-phthalanilic acid (CAS number 4727-31-5). Kartogenin can be purchased from a number of commercial suppliers, for example from Tocris under the catalog number 4513. Kartogenin induced chondrogenic differentiation of human mesenchymal stem cells, via activation of the RUNX1 pathway.

As used herein, lithium chloride (LiCl) is an ionic compound that can be used in accordance with the present invention. LiCl directly targets the TGFβ3 pathway and thereby indirectly increased the expression of the transcription factor SOX9, and aggrecan (also known as cartilage-specific proteoglycan core protein, CSPCP or chondroitin sulfate proteoglycan 1). Via TGFβ3, LiCl can also increase the amount of Type II Collagen in a cell. Lithium chloride (CAS number 7447-41-8) can be obtained from a number of commercial suppliers, for example from Sigma under the catalogue number 85144112.

As used herein, melatonin is a hormone agonist of melatonin receptors and is also known as N-acetyl-5-methoxy-tryptamine (CAS number 73-31-4). Melatonin is used to upregulate expression of BMP-2, which in turn upregulates expression of SMAD6. Melatonin can be obtained from a number of commercial suppliers, including from Tocris under the catalogue number 3550.

As used herein, rhosin hydrochloride (D-tryptophan (2E)-2-(6-quinoxalinylmethylene)hydrazide hydrochloride; CAS number 1281870-42-5) is a Rho GTPase inhibitor that inhibits binding of RhoA to guanine nucleotide exchange factors. Rhosin hydrochloride increases expression of the transcription factor SOX9, which then has a downstream effect on the expression of SOX5 and SOX 6. SOX5, SOX6 and SOX9 are all expressed during chondrogenesis and work in tandem to express chondrogenic markers. Rhosin hydrochloride can be purchased from a number of commercial suppliers, including from Tocris under the catalogue number 5003.

As used herein, leucovorin calcium (CAS no: 1492-18-8) is also known as folinic acid. Folinic acid is a 5-formyl derivative of tetrahydrofolic acid. It is readily converted to other reduced folic acid derivatives (e.g., 5,10-methylenetetrahydrofolate, 5-methyltetrahydrofolate), thus has vitamin activity equivalent to that of folic acid. Leucovorin may be used in order to increase the amount of the transcription factor PPRX1. Leucovorin calcium can be purchased from a number of commercial suppliers, including from Sigma under the catalogue number PHR1541.

As used herein, forskolin (also known as coleonol, CAS no: 66428-89-5) is a labdane diterpene that is produced by the Indian Coleus plant (Plectranthus barbatus). Other names include pashanabhedi, Indian coleus, makandi, HL-362, NKH477, and mao hou qiao rui hua. As with other members of the large diterpene family of natural products, forskolin is derived from geranylgeranyl pyrophosphate (GGPP). Forskolin contains some unique functional elements, including the presence of a tetrahydropyran-derived heterocyclic ring. Forskolin may be used for increasing the amount of the transcription factor FOSB. Forskolin can be purchased from a number of commercial suppliers, including from Tocris under the catalogue number 1099.

In certain preferred embodiments, when more than one small molecule is used in accordance with the methods of the present invention, any two, any three, any four, any five, any six or any seven of AM580, All-trans retinoic acid, 9-cis retinoic acid, β-estradiol, calcitriol, ciglitazone, kartogenin, lithium chloride, melatonin, rhosin hydrochloride, leucovorin, vorinostat, and forskolin are used. Preferably, at least AM580 (or an alternative analog of retinoic acid, or All-trans or 9-cis retinoic acid) and calcitriol are used.

In still further embodiments, the one or more small molecules may be all 8 of AM580, β-estradiol, calcitriol, ciglitazone, kartogenin, lithium chloride, melatonin, and rhosin hydrochloride.

Further still, the one or more small molecules may be all 5 of AM580, calcitriol, leucovorin, vorinostat and forskolin.

Alternatively, the one or more small molecules may be all 5 of (all-trans)-retinoic acid, 9-cis retinoic acid, calcitriol, leucovorin and vorinostat.

TABLE 5
Small molecule agents and target transcription factors
for conversion of dermal fibroblasts to chondrocytes

| Small | | Transcription | Marker/downstream |
|---|---|---|---|
| molecule | Synonym for small molecule | Factor | target |
| AM580 | 4-[(5,6,7,8-Tetrahydro-5,5,8,8-tetramethyl- | SOX9 | COL2A1 (type II |
| | 2-naphthalenyl)carboxamido] benzoic | | collagen), SOX5/6 |
| | acid; CD336; 4-((5,6,7,8-Tetrahydro- | | |
| | 5,5,8,8-tetramethyl-2- | | |
| | naphthalenyl)carbonyl)aminobenzoic acid; | | |
| | Ro-40-6055; HY-10475; LS-38310; | | |
| Retinoic acid | (2E,4E,6E,8E)-3,7-dimethyl-9-(2,6,6- | SOX9 | COL2A1 (type II |
| (including All- | trimethylcyclohexen-1-yl)nona-2,4,6,8- | | collagen), SOX5/6 |
| trans retinoic | tetraenoic acid | | |
| acid or 9-cis | | | |
| retinoic acid) | | | |
| Rhosin | MolPort-009-767-757; AKOS016367775; | SOX9 | COL2A1 (type II |
| hydrochloride | CS-3877; HY-12646; Rhosin | | collagen), SOX5/6 |
| | hydrochloride\|D-Tryptophan (2E)-2-(6- | | |
| | quinoxalinylmethylene)hydrazide | | |
| | hydrochloride | | |
| Ciglitazone | (+−)-5-(p-((1- | PPARγ | VEGFA |
| | Methylcyclohexyl)methoxy)benzyl)-2,4- | | |
| | thiazolidinedione; rac 2-Imino-5-[4-(1- | | |
| | methylcyclohexylmethoxyl)benzyl]thiazolid | | |
| | ine-4one; 2,4-Thiazolidinedione, 5-((4-((1- | | |
| | methylcyclohexyl)methoxy)phenyl)methyl)-, | | |
| | (+−)-; BML2-F01; GTPL2711; | | |
| | DTXSID0040757 | | |
| Melatonin | Melatonine; 73-31-4; N-Acetyl-5- | BMP-2 | SMAD6 |
| | methoxytryptamine; Circadin; 5-Methoxy- | | |
| | N-acetyltryptamine; Melatol; N-(2-(5- | | |
| | Methoxy-1H-indol-3-yl)ethyl)acetamide; | | |
| | N-[2-(5-Methoxy-1H-indol-3- | | |
| | yl)ethyl]acetamide; Melovin; Melatonex; | | |
| | N-[2-(5-methoxyindol-3-yl)ethyl]acetamide | | |
| Beta-estradiol | Estradiol; 17beta-Estradiol; 50-28-2; | PITX1 | SOX9 |
| | Oestradiol; Dihydrofolliculin; Estrace; | | |
| | Dihydrotheelin; Dihydroxyestrin; 3,17- | | |
| | Epidihydroxyestratriene; Estra-1,3,5(10)- | | |
| | triene-3,17beta-diol; Estradiol-3,17beta; | | |
| | B-Estradiol; 17-beta-OH-estradiol; 3,17- | | |
| | beta-Estradiol; 3,17-beta-Oestradiol; 3,17- | | |
| | Epidihydroxyoestratriene | | |
| Lithium | CHEMBL69710; CHEBI:48607; | TGFβ3 | Aggrecan, Type II |
| chloride | MFCD00011078; NSC327172 | | collagen, SOX9 |
| Calcitriol | 1alpha,25-Dihydroxyvitamin | VDR | VEGF |
| | D3; 1alpha,25-Dihydroxycholecalciferol; | | |
| | Dihydroxyvitamin D3; 1,25-DHCC; 1- | | |
| | alpha,25-Dihydroxyvitamin D3; | | |
| | 1alpha,25(OH)2D3; 1,25- | | |
| | Dihydroxycholecaliferol; 1alpha,25- | | |
| | Dihydroxyvitamin D | | |
| Kartogenin | 2-([1,1′-Biphenyl]-4-ylcarbamoyl)benzoic | RUNX1 | SOX9 |
| | acid; 2-[(4- | | |
| | phenylphenyl)carbamoyl]benzoic acid; | | |
| | CBDivE_009769; Phthalanilic acid, 4′- | | |
| | phenyl; SCHEMBL1336628; | | |
| | CHEMBL3185782 | | |
| Leucovorin | citrovorum factor, 5- | PRRX1 | TGFβ |
| | formyltetrahydrofolate, Folinic acid | | |
| Forskolin | Coleonol, pashanabhedi, Indian coleus, | FOSB | SOX9 |
| | makandi, HL-362, NKH477, mao hou qiao | | |
| | rui hua, | | |
| | (3R,4aR,5S,6S,6aS,10S,10aR,10bS)- | | |
| | 6,10,10b-Trihydroxy-3,4a,7,7,10a- | | |
| | pentamethyl-1-oxo-3-vinyldodecahydro- | | |
| | 1H-benzo[f]chromen-5-yl acetate | | |

In still further embodiments of the invention, molecules which increase the accessibility of small molecules or of transcription factors to promoter regions can be used in conjunction with the approaches of the present invention. For example, molecule which open up the chromatin may be used in conjunction with any of the combinations of transcription factors, or with any of the small molecules describes herein. One example of a molecule which can be used for opening up chromatin is vorinostat.

As used herein, vorinostat (CAS no: 149647-78-9, also known as suberanilohydroxamic acid, suberoyl+anilide+hydroxamic acid, abbreviated as SAHA and sold as “Zolinza”) is a member of a larger class of compounds that inhibit histone deacetylases (HDAC). Vorinostat also acts as a chelator for zinc ions also found in the active site of histone deacetylases. Vorinostat can be purchased from a number of commercial suppliers, including from Tocris under the catalogue number 4652.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from that of natural cells, or is otherwise flanked by a different nucleic acid sequence than that found in nature. An exogenous nucleic acid may also be extra-chromosomal, such as an episomal vector.

Screening one or more candidate agents for the ability to increase the amount of the one or more transcription factors required for conversion of a source cell type to a target cell type may include the steps of contacting a system that allows the product or expression of a transcription factor with the candidate agent and determining whether the amount of the transcription factor has increased. The system may be in vivo, for example a tissue or cell in an organism, or in vitro, a cell isolated from an organism or an in vitro transcription assay, or ex vivo in a cell or tissue. The amount of transcription factor may be measured directly or indirectly, and either by determining the amount of protein or RNA (e.g. mRNA or pre-mRNA). The candidate agent function to increase the amount of a transcription factor by increasing any step in the transcription of the gene encoding the transcription factor or increase the translation of corresponding mRNA. Alternatively, the candidate agent may decrease the inhibitory activity of a repressor of transcription of the gene encoding the transcription factor or the activity of a molecule that causes the degradation of the mRNA encoding the transcription factor or the protein of the transcription factor itself.

Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Such labelled reagents may be used in a variety of well-known assays, such as radioimmunoassays, enzyme immunoassays, e.g., ELISA, fluorescent immunoassays, and the like. See, for example, U.S. Pat. Nos. 3,766,162; 3,791,932; 3,817,837; and 4,233,402.

The methods of the invention include high-throughput screening applications. For example, a high-throughput screening assay may be used which comprises any of the assays according to the invention wherein aliquots of a system that allows the product or expression of a transcription factor are exposed to a plurality of candidate agents within different wells of a multi-well plate. Further, a high-throughput screening assay according to the disclosure involves aliquots of a system that allows the product or expression of a transcription factor which are exposed to a plurality of candidate agents in a miniaturized assay system of any kind.

The method of the disclosure may be “miniaturized” in an assay system through any acceptable method of miniaturization, including but not limited to multi-well plates, such as 24, 48, 96 or 384-wells per plate, microchips or slides. The assay may be reduced in size to be conducted on a micro-chip support, advantageously involving smaller amounts of reagent and other materials. Any miniaturization of the process which is conducive to high-throughput screening is within the scope of the invention. In any method of the invention the target cells can be transferred into the same mammal from which the source cells were obtained. In other words, the source cells used in a method of the invention can be an autologous cell, i.e., can be obtained from the same individual in which the target cells are to be administered. Alternatively, the target cell can be allogenically transferred into another individual. Preferably, the cell is autologous to the subject in a method of treating or preventing a medical condition in the individual.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Exemplary cell culture medium for use in methods of the invention are shown in Table 6.

TABLE 6
Cell culture media that can be used to culture various cell types

| Cell | Media | Cat#: | Company |
|---|---|---|---|
| Dermal | Medium106 | M-106-500 | ThermoFisher |
| fibroblasts | | | |
| H9 ESC line | KSR | 10828-028 | ThermoFisher |
| | Essential 8 | A1517001 | Life |
| | | | Technologies |
| Chondrocytes | Eagle's Minimum | 10-009-CV | Corning |
| | Essential Medium | | |

A nucleic acid or vector comprising a nucleic acid as described herein may include one or more of the sequences referred to above in Table 4 or a sequence encoding any one or more of the transcription factors listed in Tables 1 to 3.

The term “expression” refers to the cellular processes involved in producing RNA and proteins and as appropriate, secreting proteins, including where applicable, but not limited to, for example, transcription, translation, folding, modification and processing.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “vector” refers to a carrier DNA molecule into which a DNA sequence can be inserted for introduction into a host or source cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g. ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome.

The vectors described herein can be constructed and engineered using methods generally known in the scientific literature to increase their safety for use in therapy, to include selection and enrichment markers, if desired, and to optimize expression of nucleotide sequences contained thereon. The vectors should include structural components that permit the vector to self-replicate in the source cell type. For example, the known Epstein Barr oriP/Nuclear Antigen-1 (EBNA-I) combination (see, e.g., Lindner, S. E. and B. Sugden, The plasmid replicon of Epstein-Barr virus: mechanistic insights into efficient, licensed, extrachromosomal replication in human cells, Plasmid 58:1 (2007), incorporated by reference as if set forth herein in its entirety) is sufficient to support vector self-replication and other combinations known to function in mammalian, particularly primate, cells can also be employed. Standard techniques for the construction of expression vectors suitable for use in the present invention are well-known to one of ordinary skill in the art and can be found in publications such as Sambrook J, et al., “Molecular cloning: a laboratory manual,” (3rd ed. Cold Spring harbor Press, Cold Spring Harbor, N. Y. 2001), incorporated herein by reference as if set forth in its entirety.

In the methods of the invention, genetic material encoding the relevant transcription factors required for a conversion is delivered into the source cells via one or more reprogramming vectors. Each transcription factor can be introduced into the source cells as a polynucleotide transgene that encodes the transcription factor operably linked to a heterologous promoter that can drive expression of the polynucleotide in the source cell.

Suitable reprogramming vectors are any described herein, including episomal vectors, such as plasmids, that do not encode all or part of a viral genome sufficient to give rise to an infectious or replication-competent virus, although the vectors can contain structural elements obtained from one or more virus. One or a plurality of reprogramming vectors can be introduced into a single source cell. One or more transgenes can be provided on a single reprogramming vector. One strong, constitutive transcriptional promoter can provide transcriptional control for a plurality of transgenes, which can be provided as an expression cassette. Separate expression cassettes on a vector can be under the transcriptional control of separate strong, constitutive promoters, which can be copies of the same promoter or can be distinct promoters. Various heterologous promoters are known in the art and can be used depending on factors such as the desired expression level of the transcription factor. It can be advantageous, as exemplified below, to control transcription of separate expression cassettes using distinct promoters having distinct strengths in the source cells. Another consideration in selection of the transcriptional promoters is the rate at which the promoter(s) is silenced. The skilled artisan will appreciate that it can be advantageous to reduce expression of one or more transgenes or transgene expression cassettes after the product of the gene(s) has completed or substantially completed its role in the reprogramming method. Exemplary promoters are the human EF1α elongation factor promoter, CMV cytomegalovirus immediate early promoter and CAG chicken albumin promoter, and corresponding homologous promoters from other species. In human somatic cells, both EF1α and CMV are strong promoters, but the CMV promoter is silenced more efficiently than the EF1α promoter such that expression of transgenes under control of the former is turned off sooner than that of transgenes under control of the latter. The transcription factors can be expressed in the source cells in a relative ratio that can be varied to modulate reprogramming efficiency. Preferably, where a plurality of transgenes is encoded on a single transcript, an internal ribosome entry site is provided upstream of transgene(s) distal from the transcriptional promoter. Although the relative ratio of factors can vary depending upon the factors delivered, one of ordinary skill in possession of this disclosure can determine an optimal ratio of factors.

The skilled artisan will appreciate that the advantageous efficiency of introducing all factors via a single vector rather than via a plurality of vectors, but that as total vector size increases, it becomes increasingly difficult to introduce the vector. The skilled artisan will also appreciate that position of a transcription factor on a vector can affect its temporal expression, and the resulting reprogramming efficiency. As such, Applicants employed various combinations of factors on combinations of vectors. Several such combinations are here shown to support reprogramming.

After introduction of the reprogramming vector(s) and while the source cells are being reprogrammed, the vectors can persist in target cells while the introduced transgenes are transcribed and translated. Transgene expression can be advantageously downregulated or turned off in cells that have been reprogrammed to a target cell type. The reprogramming vector(s) can remain extra-chromosomal. At extremely low efficiency, the vector(s) can integrate into the cells' genome. The examples that follow are intended to illustrate but in no way limit the present invention.

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with a lipid-based transfection reagent such as Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

A number of polypeptides capable of mediating introduction of associated molecules into a cell have been described previously and can be adapted to the present invention. See, e.g., Langel (2002) Cell Penetrating Peptides: Processes and Applications, CRC Press, Pharmacology and Toxicology Series. Examples of polypeptide sequences that enhance transport across membranes include, but are not limited to, the Drosophila homeoprotein antennapedia transcription protein (AntHD) (Joliot et al., New Biol. 3: 1121-34, 1991; Joliot et al., Proc. Natl. Acad. Sci. USA, 88: 1864-8, 1991; Le Roux et al., Proc. Natl. Acad. Sci. USA, 90: 9120-4, 1993), the herpes simplex virus structural protein VP22 (Elliott and O'Hare, Cell 88: 223-33, 1997); the HIV-1 transcriptional activator TAT protein (Green and Loewenstein, Cell 55: 1179-1188, 1988; Frankel and Pabo, Cell 55: 1 289-1193, 1988); Kaposi FGF signal sequence (kFGF); protein transduction domain-4 (PTD4); Penetratin, M918, Transportan-10; a nuclear localization sequence, a PEP-I peptide; an amphipathic peptide (e.g., an MPG peptide); delivery enhancing transporters such as described in U.S. Pat. No. 6,730,293 (including but not limited to an peptide sequence comprising at least 5-25 or more contiguous arginines or 5-25 or more arginines in a contiguous set of 30, 40, or 50 amino acids; including but not limited to an peptide having sufficient, e.g., at least 5, guanidino or amidino moieties); and commercially available Penetratin™ 1 peptide, and the Diatos Peptide Vectors (“DPVs”) of the Vectocell® platform available from Daitos S. A. of Paris, France. See also, WO/2005/084158 and WO/2007/123667 and additional transporters described therein. Not only can these proteins pass through the plasma membrane but the attachment of other proteins, such as the transcription factors described herein, is sufficient to stimulate the cellular uptake of these complexes.

The present invention includes the following non-limiting Examples.

### EXAMPLES

### Example 1

Candidate transcription factors for conversion of fibroblasts to chondrocytes were identified according to the methods previously described in Rackham et al (2016) Nature Genetics, 48(3): 331-335.

Fibroblasts were expanded in a monolayer (2D) under normoxic or hypoxic conditions. The experiment was performed under both conditions, since cartilage is known to be a particularly hypoxic environment, due to the absence of significant vasculature. Performing the experiment under hypoxic conditions indicates that the methodology may be extrapolated to in vivo treatment.

A mixture of small molecules, as shown in Table 7 below, was added to the fibroblast culture, at the concentrations specified in the table:

TABLE 7
Small molecules used to convert dermal fibroblasts to chondrocytes

| | Final | | | Catalogue |
|---|---|---|---|---|
| Small molecule | concentration | | Company | number |
| AM580 | 1 | μM | Tocris | #0760 |
| Rhosin hydrochloride | 30 | μM | Tocris | # 5003 |
| Ciglitazone | 5 | μM | Tocris | #1307 |
| Melatonin | 50 | nM | Tocris | #3550 |
| Beta-estradiol | 1 | nM | Tocris | #2824 |
| Lithium chloride | 5 | μM | Sigma | L7026 |
| Calcitriol | 60 | nM | Tocris | #2551 |
| Kartogenin | 100 | μM | Tocris | #4513 |

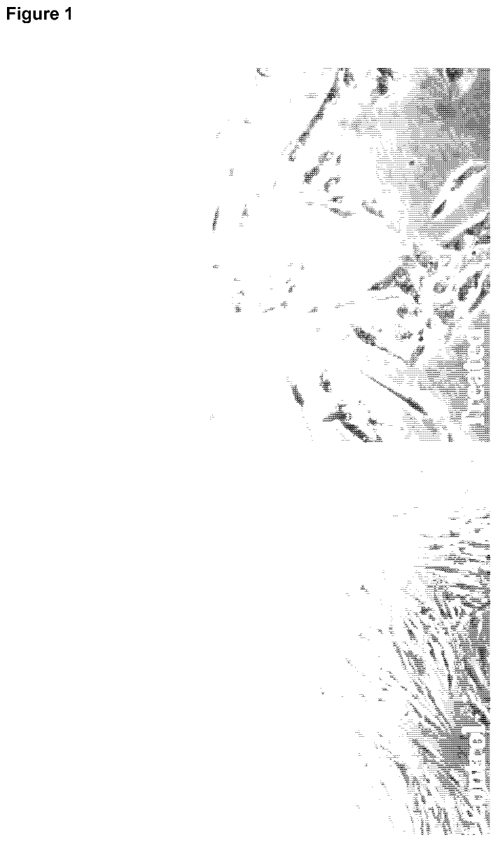

Over a period of 10 days, the fibroblasts changed macroscopic appearance and assumed the typical cuboidal appearance associated with isolated native chondrocytes in culture (see FIG. 1).

### Example 2

Gene expression studies were conducted to confirm the conversion at the molecular level. Converted fibroblasts from Example 1 were harvested and RNA was extracted to determine the expression of SOX9, a transcription factor that drives downstream upregulation of chondrogenic genes (such as aggrecan and type II collagen).

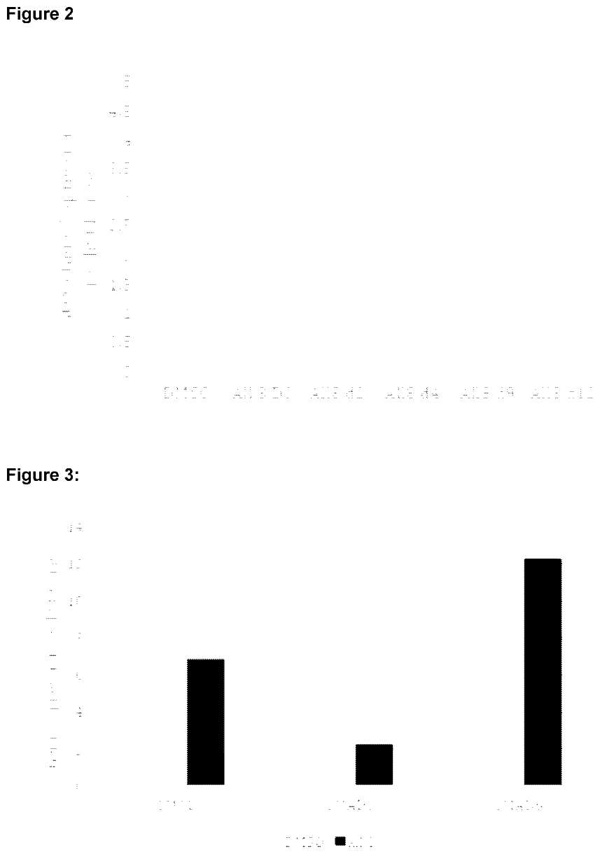

The results of gene expression experiments confirm the conversion of the fibroblasts to a chondrocytic phenotype. FIG. 2 shows the gradual upregulation of SOX9 in the treated cells, peaking at 9 days and stabilising at that level for at least 3 more days (until the conclusion of the experiment).

The upregulation of SOX9 during chondrogenesis of stem cells is driven by an upstream signalling family of proteins called SMAD. The expression of members of the SMAD family was determined in fibroblasts cultured for 14 days in 2D with the combination of small molecules shown in Table 7. The results shown in FIGS. 2 and 3 show increased expression of SMAD 3, SMAD 5 and SMAD6, indicating that the converted cells are developing into mature chondrocytes.

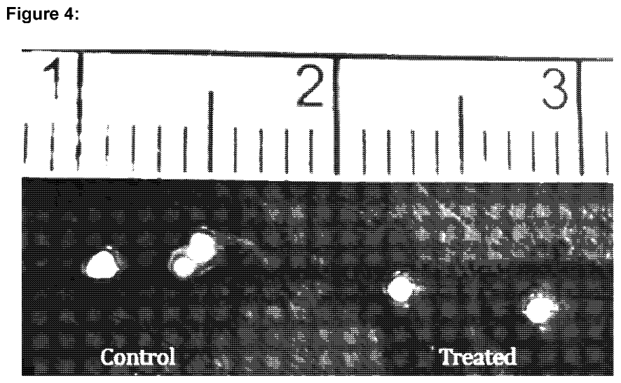

Driving chrondrogenesis requires moving from 2D to 3D cultures. The condensation of chrondrogenic cells that takes place during development is a critical step in driving the maturation of chondrocytes. To determine whether condensation had occurred with the converted chondrocytes, cells grown for 14 days in 2D culture were pelleted by centrifugation and cultures for a further 21 days. The pellets appeared to amass white extracellular matrix that appear macroscopically, and on touch, to be rigid (similarly to native cartilage). These features were only observed in the treatment group of cells, but not in the control group, which were soft and dispersed (see FIG. 4).

The cell pellets of the treated cells (now displaying characteristics of chondrocytes) were subjected for further analysis by histology. Cells were stained for broad matrix deposition by Hematoxylin and Eosin (H&E staining) and for markers of chondogenic differentiation (deposition of aggrecan and type II collagen). Staining to detect presence of the fibroblast marker, type I collagen was also conducted to evaluate the loss of donor cell phenotype.

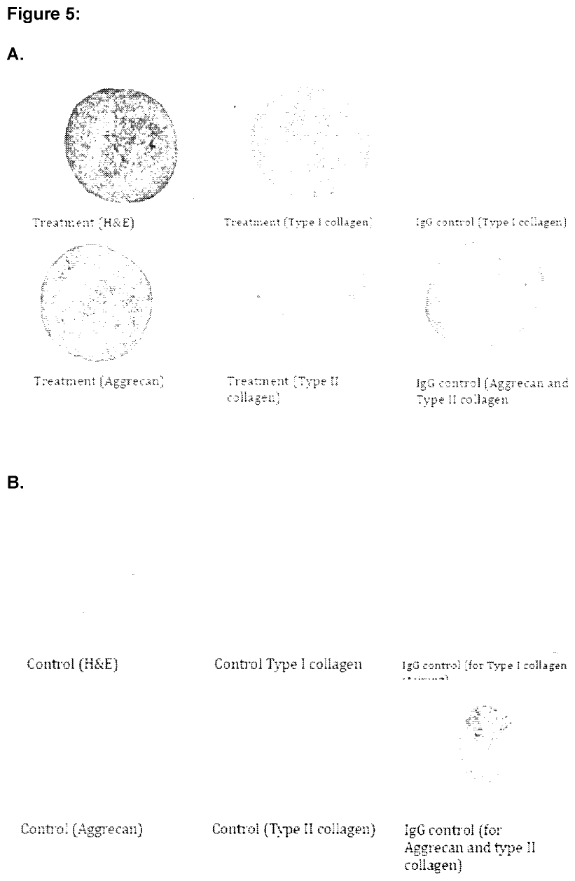

FIG. 5A shows the histology results for control (untreated) cells. The cell pellets appear small with little matrix deposition. Staining for chondrogenic markers was negative but positive for type I collagen, indicating a lack of conversion from a fibroblast cell type to chondrocyte. FIG. 5B shows the results for treated cells. The cell pellets are larger than for the control cells, and are extensively stained for extracellular matrix. Importantly, the cells stained positively for aggrecan and type II collagen

### Example 3

Chondrocytes were obtained from human cartilage. Cells were expanded in 2D through several passages to induce de-differentiation (to replicate diseased or de-differentiated chondrocytes as arises in osteoarthritis or in typical passaging of primary chondrocytes in vitro).

Expanded cells were incubated with or without the small molecules listed in Example 1, for 2 weeks in 2D culture under normoxic and hypoxic conditions.

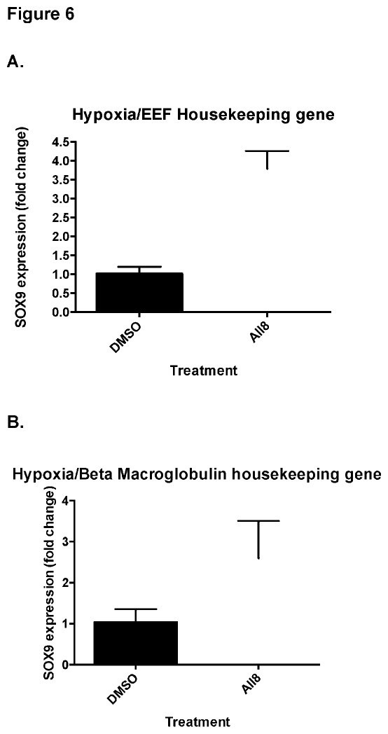

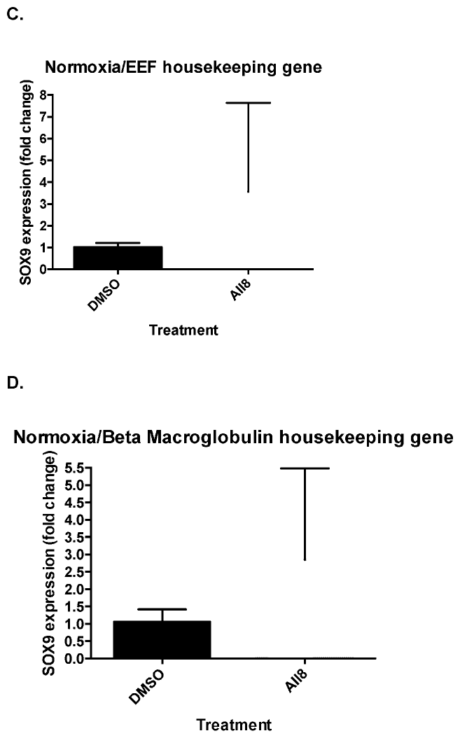

Gene expression analysis was conducted on cells to determine whether re-differentiation had occurred. SOX9 gene expression indicated that the de-differentiated chondrocytes had been converted into “native” or re-differentiated chondrocytes (FIG. 6).

Conclusion:

A set of small molecules has been identified, which can be used to increase expression of relevant transcription factors and to robustly convert fibroblasts into chondrocytes. The same molecules are also useful for converting de-differentiated chondrocytes into re-differentiated chondrocytes.

### Example 4

Human Dermal Fibroblasts were seeded onto well plates at 7×10⁴ cells/cm² 24 hours prior to viral transduction of transcription factors in medium 106 with LSGS (Life Technologies). On the day following seeding, lentiviral particles encoding the transcription factors BARX1, PITX1, SMAD6, FOXC1, SIX2, AHR and JUNB and IRES-GFP were transduced to the cells in Medium 106 with Polybrene (Merck Millipore). Well plates were then centrifuged at 1900 rpm for 60 minutes immediately after transduction. At day 2, medium was replaced with chondrocyte differentiation medium (DMEM (Life Technologies), 10% FBS (Life Technologies), 1× Non-essential amino acids (Life Technologies), 100 U/ml Penicillin Streptomycin (Life Technologies), 5 ng/ml BFGF (Miltenyi Biotec)). Medium was changed every 2 days throughout the experiment. The experiment was conducted in 2D and finished at day 14.

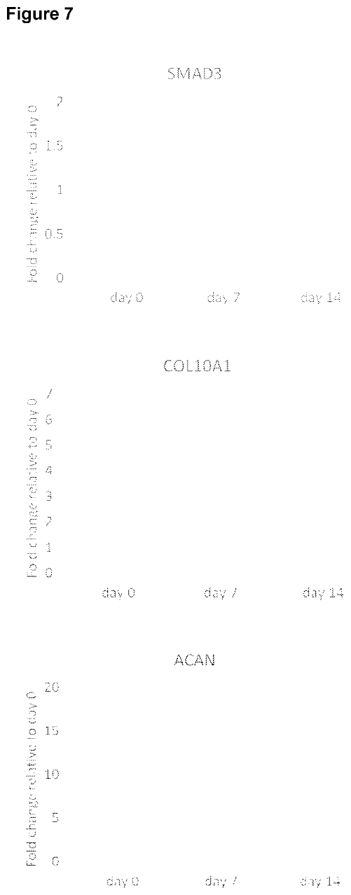

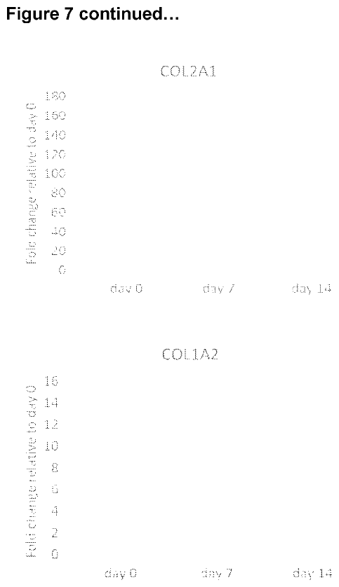

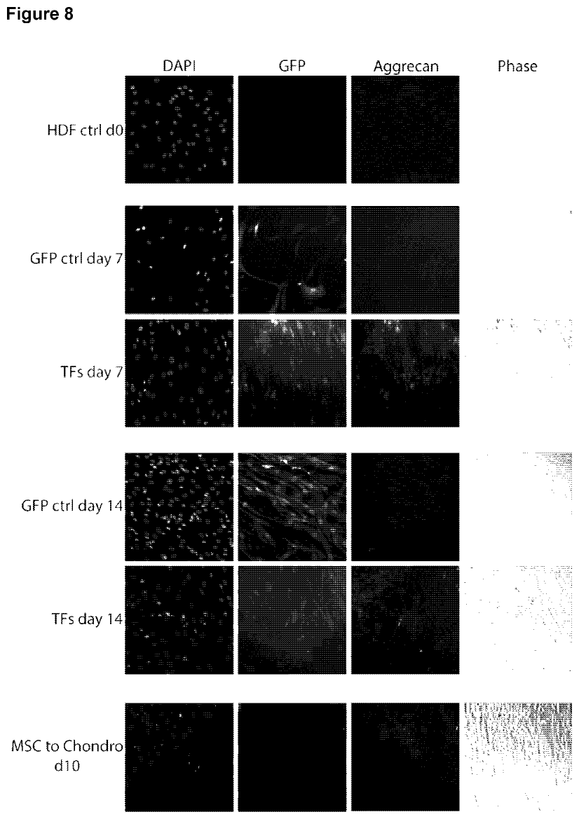

At days 0, 7 and 14, after transduction, gene expression of chondrocyte markers was performed to monitor conversion of the fibroblasts to chondrocytes (FIG. 7). At the same time points, cells were stained for aggrecan (a chondrocyte marker) (FIG. 8). The data demonstrate successful conversion of the fibroblasts to chondrocytes (for example, the presence of positive staining for aggrecan in cells transduced with the transcription factors in FIG. 8).

### Example 5

Human dermal fibroblasts were seeded and transfected with lentiviral particles encoding doxycycline-inducible expression of transcription factors using a similar method to that described for Example 4. The transcription factors were BARX1, PITX1, SMAD6, FOXC1, SIX2, AHR, FOSB and JUNB. Transcription factor expression was induced using doxycycline. Lentiviral particles encoding EGFP were used as a negative control.

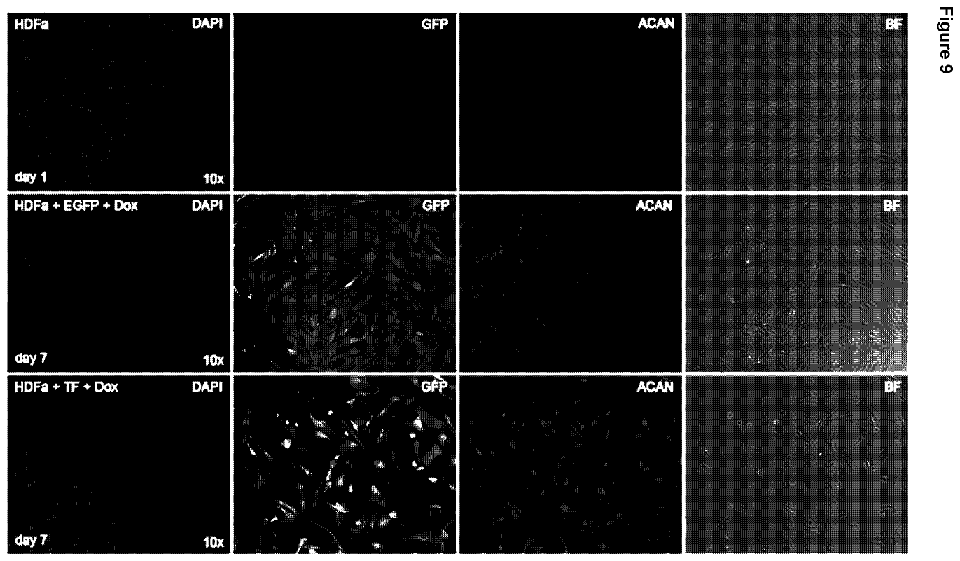

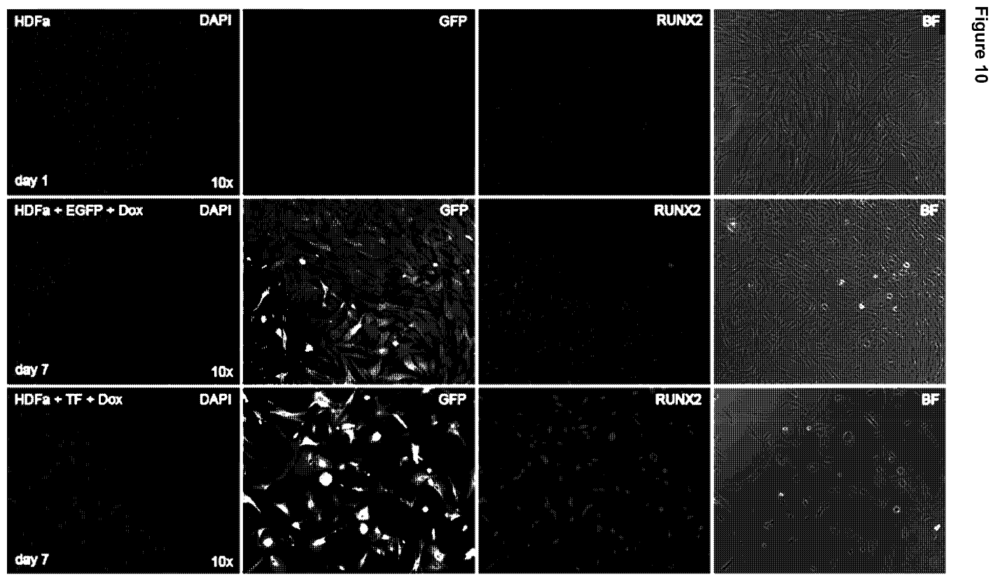

At Day 7, cells were stained for aggrecan and RUNX2 (both markers of chondrocyte differentiation) (see FIGS. 9 and 10). Compared to controls, there was a significant upregulation of aggrecan and of RUNX2 upon expression of the transcription factors.

### Example 6

Chondrocytes obtained from non-damaged human cartilage were incubated in vitro with small molecules similarly to the method described in Example 3. DMSO (vehicle) was included as a negative control. TGFβ was used as a positive control as this growth factor is known to drive chondrogenic differentiation.

Small molecule mixtures were as described in Table 7 (“All 8”), or as set out further below:

TABLE 8
Alternative small molecule mixture E

| | Final | | | Catalogue |
|---|---|---|---|---|
| Small molecule | concentration | | Company | number |
| AM580 | 1 | μM | Tocris | 0760 |
| Calcitriol | 60 | nM | Tocris | 2551 |
| Leucovorin calcium | 2 | μM | Sigma | PHR1541 |
| (folate) | | | | |
| Vorinostat | 10 | μM | Tocris | 4652 |
| Forskolin | 5 | μM | Tocris | 1099 |

TABLE 9
Alternative small molecule mixture F

| | Final | | | Catalogue |
|---|---|---|---|---|
| Small molecule | concentration | | Company | number |
| (All-trans)-Retinoic | 1 | μM | Sigma | R2625 |
| Acid | | | | |
| 9-cis Retinoic acid | 1 | μM | Sigma | R4643 |
| Calcitriol | 60 | nM | Tocris | 2551 |
| Vorinostat | 10 | μM | Tocris | 4652 |
| Leucovorin calcium | 2 | μM | Sigma | PHR1541 |
| (folate) | | | | |

### Example 7

Expanded cells were incubated with or without the small molecules listed in Tables 7 (“all8”), 8 (Combination E) and 9 (combination F) for 2 weeks in 2D culture under normoxic and hypoxic conditions.

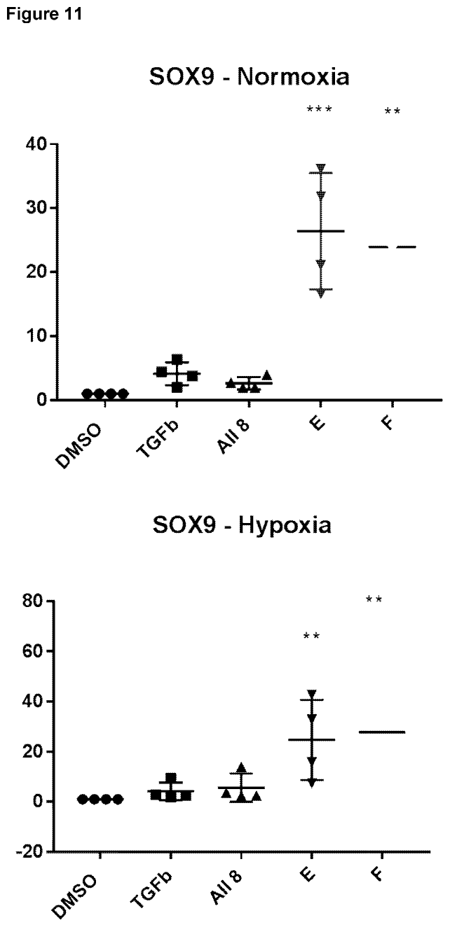

Gene expression analysis was conducted on cells to determine whether re-differentiation had occurred. SOX9 gene expression indicated that the de-differentiated chondrocytes had been converted into “native” or re-differentiated chondrocytes (FIG. 11). The small molecules were more effective than TGFβ in increasing the levels of this marker of chrondrocyte differentiation.

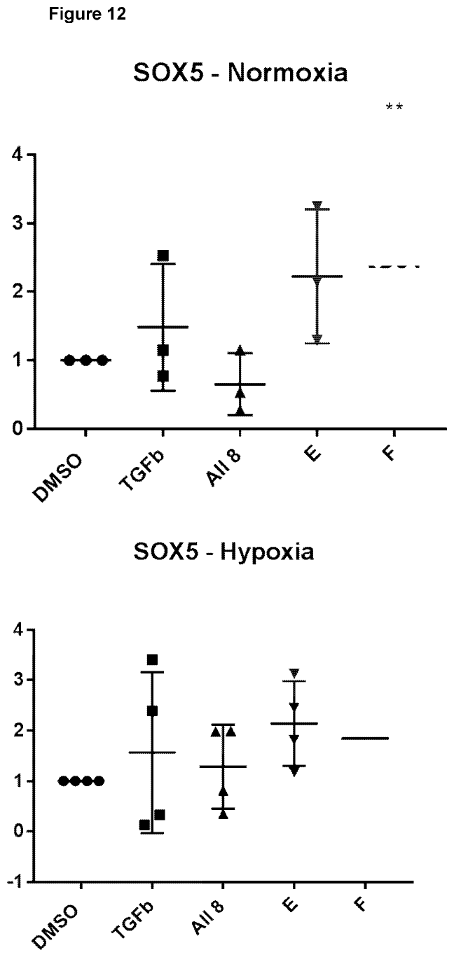

SOX5 gene expression was also increased upon incubation with small molecule mixtures E and F compared to negative controls. The increase in expression of this marker was more evident under normoxic conditions than hypoxic conditions (FIG. 12).

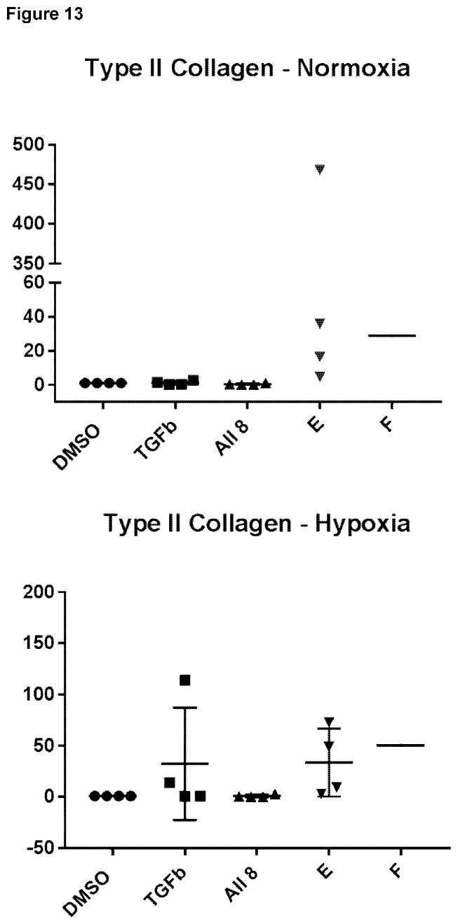

In addition, the expression of type II collagen was increased to a greater extent by the small molecules than by TGFβ (FIG. 13).

Preliminary results from cell obtained from one individual indicate that expression of aggrecan also increases in hypoxic conditions following incubation with the mixtures identified in Tables 8 and 9.

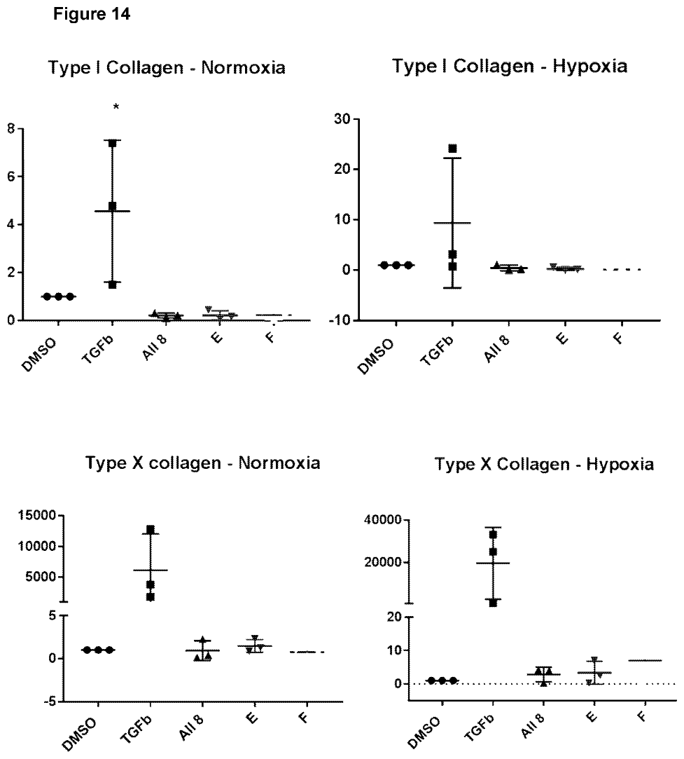

In contrast to TGFβ, the small molecules tested in Example 1 (“All 8”) and as listed in Tables 8 and 9 all decreased expression of type 1 collagen (a marker of undifferentiated or de-differentiated chondrocytes) and of Type X collagen (a hypertrophic factor commonly expressed in osteoarthritis) (FIG. 14).

### Example 8

Chondrocytes were isolated from tissue removed from a patient. Cells were seeded into flasks and allowed to adhere/acclimatise for 48 hours. Medium was replaced with cell conversion medium containing small molecules, for 9 days.

TGFβ was included in media as a positive control as this growth factor is known to drive chondrogenic differentiation.

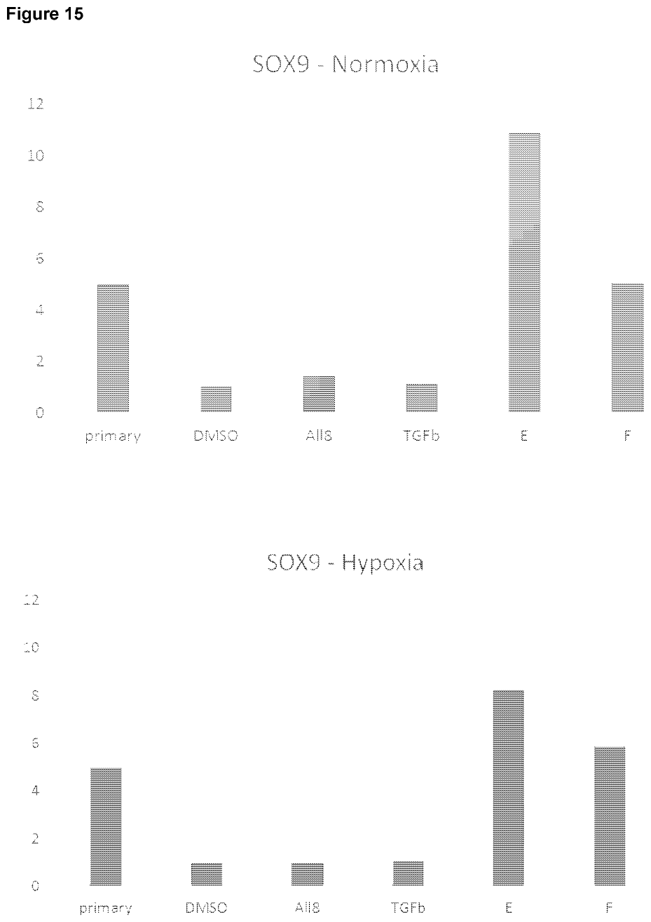

The results (FIG. 15) indicate that the use of the small molecules in cell medium could prevent de-differentiation of primary chondrocytes.

1-55. (canceled)

56. A method for reprogramming a source cell to produce a cell that exhibits at least one characteristic of a chondrocyte comprising:
i) providing a source cell or a cell population comprising a source cell;
ii) contacting said source cell with one or more agents that activate or increase the expression of two or more transcription factors; and
iii) culturing said cell or cell population, and optionally monitoring the cell or cell population for at least one characteristic of a chondrocyte cell, wherein the transcription factors are two or more of those listed in Tables 1 to 4.

57. The method according to claim 56, wherein the source cell is a pluripotent, multipotent or somatic cell.

58. The method according to claim 57, wherein the pluripotent cell is an embryonic stem cell.

59. The method according to claim 57, wherein the source cell is a fibroblast, dermal fibroblasts or de-differentiated chondrocyte.

60. The method according to claim 56, wherein the source cell is the dermal fibroblast and the transcription factors are any two or more of the transcription factors listed in Tables 1 or 4.

61. The method according to claim 56, wherein the source cell is the de-differentiated chondrocyte and the transcription factors are any two or more of the transcription factors listed in Tables 3.

62. A method of generating a cell exhibiting at least one characteristic of a chondrocyte from a de-differentiated chondrocyte, the method comprising:
increasing the amount of two or more transcription factors, or variants thereof, in a de-differentiated chondrocyte; and
culturing the de-differentiated chondrocyte for a sufficient time and under conditions to allow re-differentiation to a chondrocyte, wherein the transcription factors are two or more of those listed in Table 3.

63. The method of claim 62, wherein the protein expression or amount of the transcription factors as shown in a single row of Table 3 is increased.

64. A method of preventing the de-differentiation of a chondrocyte cell culture, the method comprising:
providing a de-differentiated chondrocyte cell, or a cell population comprising a de-differentiated chondrocyte cell;
contacting said de-differentiated chondrocyte cell with one or more agents that activate or increase the expression of two or more transcription factors; and
culturing said de-differentiated cell or cell population, and optionally monitoring the cell or cell population for at least one characteristic of a chondrocyte cell thereby preventing the de-differentiation of the chondrocyte and maintaining at least one characteristic of a chondrocyte in the chondrocyte cell culture, wherein the transcription factors are two or more of those listed in Table 3.

65. The method of claim 56, wherein the protein expression or amount of the following transcription factors is increased:
a) IL11, SOX5, VEGFA, RUNX1, VDR, NR2F1, FOSB and PRRX1;
b) SOX9, TCF4, VEGFA, RUNX1, VDR, NR2F1, FOSB and PRRX1;
c) SOX9, SOX5, HOXA7, RUNX1, VDR, NR2F1, FOSB and PRRX1;
d) SOX9, SOX5, VEGFA, TCF4, VDR, NR2F1; FOSB and PRRX1;
e) SOX9, SOX5, VEGFA, RUNX1, TCF4, NR2F1, FOSB and PRRX1;
f) SOX9, SOX5, VEGFA, RUNX1, VDR, HOXA7, FOSB and PRRX1;
g) SOX9, SOX5, VEGFA, RUNX1, VDR, NR2F2, FOSB and PRRX1;
h) SOX9, SOX5, VEGFA, RUNX1, VDR, NR2F1, HOXA7 and PRRX1; or
i) SOX9, SOX5, VEGFA, RUNX1, VDR, NR2F1, FOSB and HOXA7;

66. The method of claim 59, wherein the de-differentiated chondrocyte is obtained from an expanded culture of chondrocytes, or is obtained from a biological sample from an individual suffering from osteoarthritis.

67. The method of claim 56, wherein the transcription factors are two or more of SOX9, SOX5, VEGFA, RUNX1, VDR, NR2F1, FOSB and PRRX1.

68. The method of claim 56, wherein the transcription factors are SOX9, SOX5, VEGFA, RUNX1, VDR, NR2F1, FOSB and PRRX1.

69. The method of claim 56, wherein protein expression or the amount of the two or more transcription factors, or variants thereof, is increased in a source cell by contacting the source cell with an agent that increases the expression or amount of the two or more transcription factors, preferably wherein the agent is selected from the group consisting of: a nucleic acid, a protein, an aptamer, a small molecule, ribosome, an RNAi agent and peptide-nucleic acid (PNA), and analogues or variants thereof, and more preferably wherein the agent is a nucleic acid encoding one or more transcription factors listed in any one of Tables 1 to 4.

70. The method of claim 56, wherein the protein expression or amount of the two or more transcription factors is increased in a source cell by contacting the source cell with one or more small molecules that increase the expression or amount of the two or more transcription factors, preferably wherein the one or more small molecules is selected from the group consisting of: [4-[(5,6,7,8-Tetrahydro-5,5,8,8-tetramethyl-2-naphthalenyl)carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin, vorinostat and forskolin.

71. The method of claim 70, wherein the source cell is contacted with [4-[(5,6,7,8-Tetrahydro-5,5,8,8-tetramethyl-2-naphthalenyl)carboxamido]benzoic acid (AM580), beta-estradiol, calcitriol, ciglitazone, kartogenin, lithium chloride, melatonin, and rhosin hydrochloride.

72. The method of claim 70, wherein the source cell is contacted with [4-[(5,6,7,8-Tetrahydro-5,5,8,8-tetramethyl-2-naphthalenyl)carboxamido]benzoic Acid (AM580), calcitriol, leucovorin, vorinostat and forskolin.

73. The method of claim 70, wherein the source cell is contacted with all-trans retinoic acid, 9-cis retinoic acid, calcitriol, leucovorin and vorinostat.

74. The method of claim 56, wherein culturing the source cell for a sufficient time and under conditions to allow differentiation to a chondrocyte includes culturing the cells for at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days in a relevant medium as shown in Table 6, preferably wherein the culturing of the cells is performed under hypoxic conditions.

75. The method of claim 56, wherein the method further includes the step of administering the cell exhibiting at least one characteristic of a chondrocyte to an individual.

76. A cell exhibiting at least one characteristic of a chondrocyte, wherein the cell is produced by the method of claim 56.

77. A population of cells, wherein at least 5% of cells exhibit at least one characteristic of a chondrocyte, wherein the cells are produced by the method of claim 56.

78. The method of claim 56, wherein the at least one characteristic of a chondrocyte is selected from a morphological marker, the expression of one or more genes or the production of one or more peptides or protein/polysaccharide complexes.

79. The population of cells of claim 77, for use in a method of treating osteoarthritis or other condition characterized by degeneration of cartilage tissue.

80. One or more of [4-[(5,6,7,8-Tetrahydro-5,5,8,8-tetramethyl-2-naphthalenyl)carboxamido]benzoic acid (AM580), all-trans retinoic acid, 9-cis retinoic acid, beta-estradiol, calcitriol, ciglitazone, kartogenin, lithium chloride, melatonin, rhosin hydrochloride, leucovorin, vorinostat and forskolin, for use in a method of treating osteoarthritis or other condition characterized by degeneration of cartilage tissue.

81. A composition comprising at least one chondrocyte and at least one agent which increases the activity or the protein expression of two or more transcription factors in the chondrocyte, wherein the transcription factor may be any of the transcription factors listed in Tables 1 to 4.
